(19) United States
(12) Patent Application Publication
(10) Pub. No.: US 2026/0060576 A1
(43) Pub. Date: Mar. 5, 2026

(54) WEARABLE DEVICE COMPRISING SENSOR

(71) Applicant: SAMSUNG ELECTRONICS CO., LTD., Suwon-si (KR)

(72) Inventor: Dongjin KIM, Suwon-si (KR)

(73) Assignee: SAMSUNG ELECTRONICS CO., LTD., Suwon-si (KR)

(21) Appl. No.: 19/381,520

(22) Filed: Nov. 6, 2025

Related U.S. Application Data
(63) Continuation of application No. PCT/KR2024/005180, filed on Apr. 17, 2024.

(30) Foreign Application Priority Data
Jun. 27, 2023 (KR) 10-2023-0082576
Jul. 20, 2023 (KR) 10-2023-0094948
Aug. 29, 2023 (KR) 10-2023-0114044

(51) Int. Cl. A61B 5/1455; A61B 5/00

(52) U.S. Cl. CPC A61B 5/14552; A61B 5/6802; A61B 2562/0233

(57) ABSTRACT

A wearable device includes: an insulating member having a first surface contacting with a portion of a body of a user wearing the wearable device; and an optical sensor surrounded by the insulating member and configured to detect biometric information of the user, wherein the first surface includes: a first region; and a second region surrounded by the first region, and wherein the second region includes a Fresnel pattern aligned to the optical sensor and the second region is recessed relative to the first region.

### CROSS-REFERENCE TO RELATED APPLICATIONS

This application is a by-pass continuation application of International Application No. PCT/KR2024/005180, filed on Apr. 17, 2024, which is based on and claims priority to Korean Patent Application Nos. 10-2023-0082576, filed on Jun. 27, 2023, 10-2023-0094948, filed on Jul. 20, 2023, and 10-2023-0114044, filed on Aug. 29, 2023, in the Korean Intellectual Property Office, the disclosures of which are incorporated by reference herein their entireties.

### BACKGROUND

### 1. Field

The present disclosure relate to a wearable device including a sensor.

### 2. Description of Related Art

A wearable device has changed a way a user monitors health. The wearable device typically worn on a wrist is becoming increasingly popular due to a function capable of tracking various biometric parameters such as a heart rate, a sleep pattern, and an activity level. However, alternative wearable form factors that may provide an improved function are required, and a ring-type wearable device with a biometric sensor is being developed to address these requirements.

The above-described information may be provided as related art for a purpose of helping understanding of the present disclosure. No claim or determination is raised as to whether any of the above-described descriptions may be applied as prior art related to the present disclosure.

### SUMMARY

According to an aspect of the disclosure, a wearable device includes: an insulating member having a first surface contacting with a portion of a body of a user wearing the wearable device; and an optical sensor surrounded by the insulating member and configured to detect biometric information of the user, wherein the first surface includes: a first region; and a second region surrounded by the first region, and wherein the second region includes a Fresnel pattern aligned to the optical sensor and the second region is recessed relative to the first region.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates an exemplary wearable device according to an embodiment;

FIG. 2 is an exploded view of an exemplary wearable device according to an embodiment;

FIG. 3 is a cross-sectional view indicating an exemplary wearable electronic device according to an embodiment;

FIG. 4 is a diagram indicating an exemplary wearable device according to an embodiment.

FIG. 5 is a diagram for describing an exemplary first pattern according to an embodiment;

FIG. 6 is a diagram indicating an exemplary wearable device according to an embodiment;

FIG. 7 is a diagram for describing an exemplary second pattern according to an embodiment;

FIG. 8 is a diagram indicating an exemplary wearable device according to an embodiment;

FIG. 9 is a diagram indicating a pattern of exemplary grooves according to an embodiment;

FIG. 10 is a diagram indicating a pattern of exemplary grooves according to an embodiment;

FIG. 11 is a diagram illustrating a pattern of exemplary grooves according to an embodiment;

FIG. 12 is a cross-sectional view indicating an exemplary wearable electronic device according to an embodiment;

FIG. 13 is a diagram for describing a pattern area of a light receiving portion and a light emitting portion according to an embodiment;

FIG. 14 is a diagram for describing a pattern area of a light receiving portion and a light emitting portion according to an embodiment;

FIG. 15A is a diagram indicating an exemplary electronic device according to an embodiment;

FIG. 15B is a cross-sectional view cut along line A-A′ of FIG. 15A;

FIG. 15C is a diagram exemplarily indicating the pattern of FIG. 15A;

FIG. 15D is a diagram exemplarily indicating the pattern of FIG. 15A;

FIG. 16A is a diagram indicating an exemplary electronic device according to an embodiment;

FIG. 16B is a cross-sectional view cut along line B-B′ of FIG. 16A;

FIG. 17 is a diagram indicating an exemplary electronic device according to an embodiment;

FIG. 18A is a diagram indicating an exemplary electronic device according to an embodiment;

FIG. 18B is a cross-sectional view cut along line C-C′ of FIG. 18A;

FIG. 19A is a cross-sectional view indicating an exemplary electronic device according to an embodiment;

FIG. 19B is a cross-sectional view indicating an electronic device according to a comparative example;

FIG. 19C is a cross-sectional view indicating an electronic device according to a comparative example;

FIG. 20 is a diagram indicating an example of an electronic device according to an embodiment and electronic devices according to a comparative example;

FIG. 21 is a diagram indicating an exemplary electronic device according to an embodiment; and

FIG. 22 is a block diagram of an electronic device in a network environment according to various embodiments.

### DETAILED DESCRIPTION

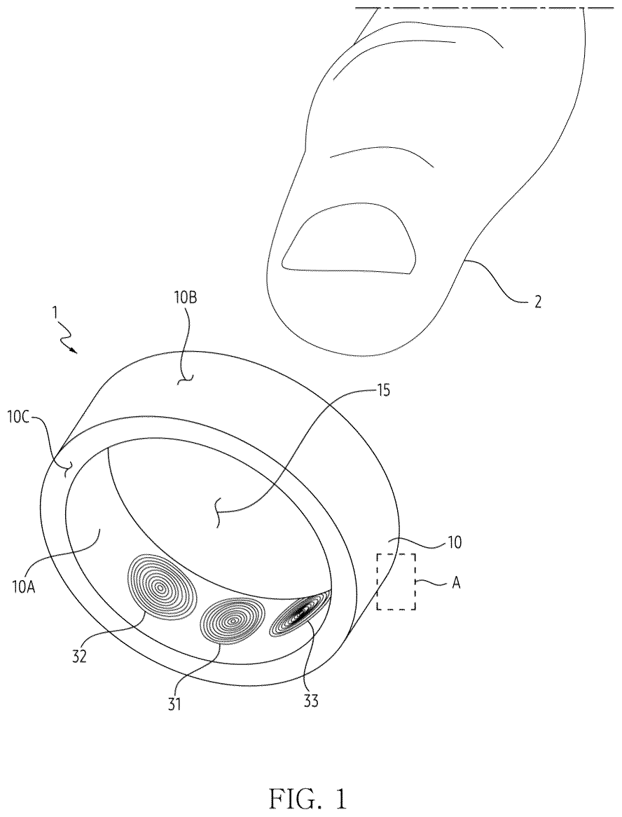

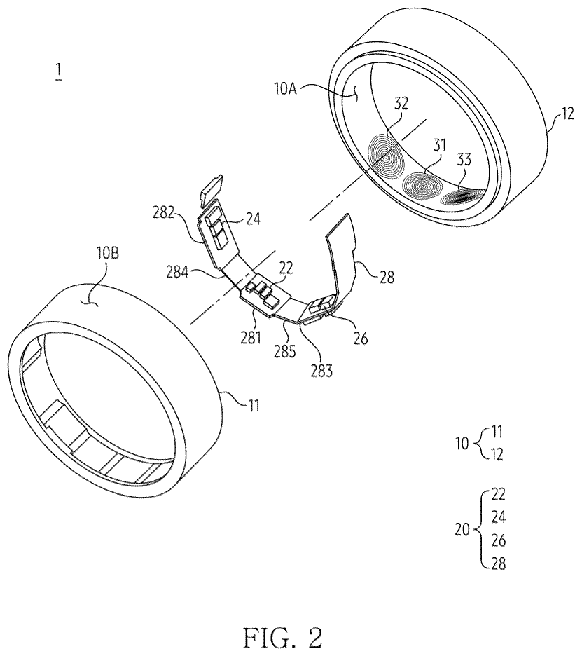

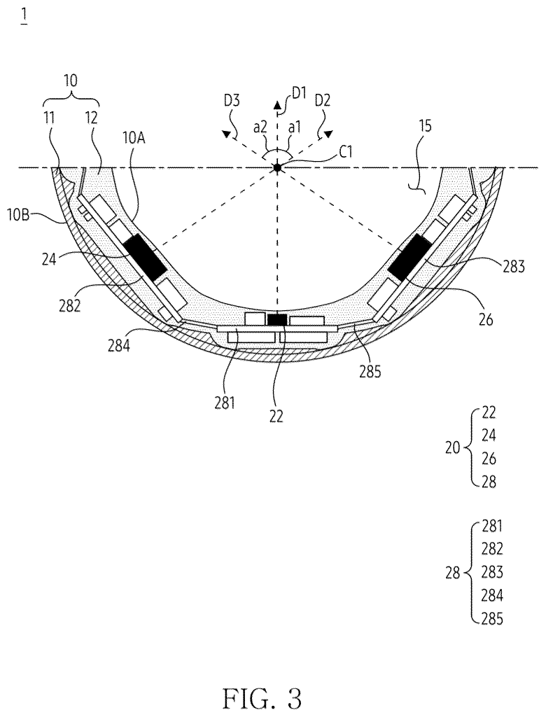

FIG. 1 illustrates an exemplary wearable device according to an embodiment. FIG. 2 is an exploded view of an exemplary wearable device according to an embodiment. FIG. 3 is a cross-sectional view indicating an exemplary wearable electronic device according to an embodiment. FIG. 3 indicates a cross-section cut along a surface A of FIG. 1.

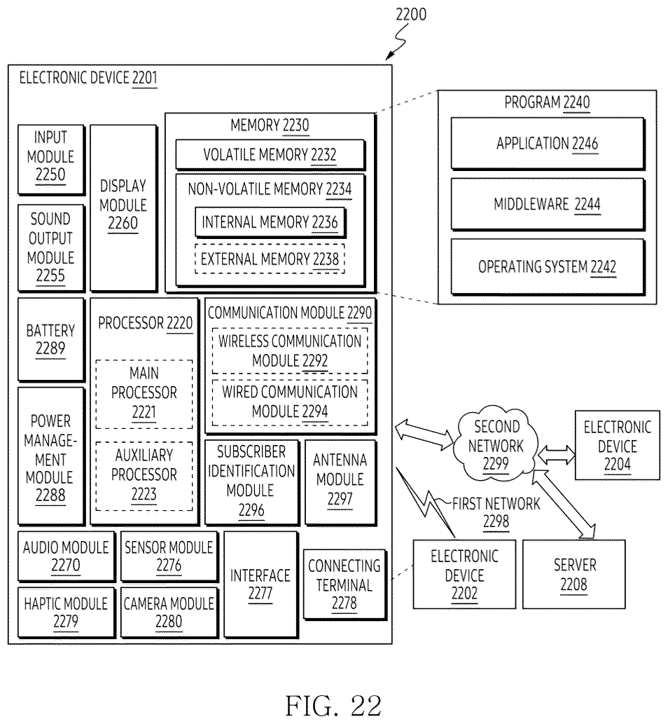

Referring to FIGS. 1, 2, and 3, a wearable device 1 (e.g., the electronic device 2201 of FIG. 22) according to an embodiment may be configured to be wearable by a user. For example, the wearable device 1 may have a ring shape with a hole 15 provided so that the user may insert a body (or a portion of a body) 2 (e.g., a finger). However, the present disclosure is not limited to the above example embodiment, and the wearable device 1 may have various shapes corresponding to the body to be worn on the body of the user.

The wearable device 1 according to an embodiment may include a housing 10 and a sensor module 20 in the housing 10. The housing 10 may include a frame 11 and an insulating member 12. The sensor module 20 may include a light emitting portion 22, a first light receiving portion 24, and a second light receiving portion 26. The sensor module 20 may further include a substrate 28 on which the light emitting portion 22, the first light receiving portion 24, and the second light receiving portion 26 are disposed.

In an embodiment, the housing 10 may form an exterior of the wearable device 1. For example, the housing 10 may form or define a first surface 10A, a second surface 10B, and a third surface 10C. When the user wears the wearable device 1, the first surface 10A may surround the body of the user to face the body 2 of the user. The first surface 10A may be at least partially in contact with the body 2 of the user. For example, a first area 31, a second area 32, and a third area 33 formed on the first surface 10A may be in contact with the body 2 of the user. The first area 31, the second area 32, and the third area 33 may be spaced apart from each other. The first area 31 may be positioned between the second area 32 and the third area 33. Light emitted from the light emitting portion 22 may be transmitted to the body 2 of the user through the first area 31. The light reflected from the body 2 of the user may reach the first light receiving portion 24 and the second light receiving portion 26 through the second area 32 and the third area 33. The second surface 10B may be spaced apart from the first surface 10A and may face an opposite direction to the first surface 10A. The third surface 10C may surround a space between the first surface 10A and the second surface 10B. For example, the third surface 10C may extend from a periphery of the first surface 10A to a periphery of the second surface 10B. The hole 15 defined by the first surface 10A may be formed in the housing 10 to accommodate the body 2 of the user. The first surface 10A may be or correspond to an inner circumferential surface. The second surface 10B may be or correspond to an outer circumferential surface.

In an embodiment, the insulating member 12 of the housing 10 may at least partially form the first surface 10A. For example, the insulating member 12 may form at least the first area 31, the second area 32, and the third area 33 of the first surface 10A. For example, the insulating member 12 may form the first area 31, the second area 32, and the third area 33, and may also form a portion of the first surface 10A extending from the first area 31, the second area 32, and the third area 33. The second surface 10B and the third surface 10C may be formed by a frame 11 of the housing 10, but the present disclosure is not limited to the above example embodiment. For example, the second surface 10B and/or the third surface 10C may be formed by the frame 11 and the insulating member 12 together.

In an embodiment, the frame 11 of the housing 10 may be formed of metal and/or plastic. The frame 11 and the insulating member 12 may be coupled to each other. For example, through a process such as insert injection, the insulating member 12 and the frame 11 may be coupled. For example, the insulating member 12 coupled to the frame 11 may be formed by injecting a molten resin into a mold in which the frame 11 is disposed, but is not limited to the above example. The insulating member 12 may include, for example, the resin such as epoxy, but is not limited to the above example.

The frame 11 may be or correspond to a first frame, a first housing, or a first support member. The insulating member 12 may be or correspond to a second frame, a second housing, a second support member, or a non-conductive member. The first to third areas 31, 32, and 33 may be or correspond to first to third sensing areas, first to third pattern areas, first to third Fresnel pattern areas, or first to third condensing areas, respectively.

In an embodiment, the sensor module 20 may be disposed in the housing 10. The sensor module 20 may be positioned between the first surface 10A and the second surface 10B. The sensor module 20 may be at least partially surrounded by the insulating member 12. The sensor module 20 may be at least partially hemmed in by an insulating member. For example, the insulating member 12 may surround the sensor module 20 disposed on the frame 11. Accordingly, the frame 11 and/or the insulating member 12 may support the sensor module 20 and fix the sensor module 20 in the housing 10.

In an embodiment, the sensor module 20 may be at least partially attached on the frame 11. For example, a first support portion 281, a second support portion 282, and a third support portion 283 of the substrate 28 may be attached on the frame 11, but are not limited thereto.

In an embodiment, the substrate 28 may include the first support portion 281, the second support portion 282, the third support portion 283, a first connection portion 284, and a second connection portion 285. The light emitting portion 22 may be disposed on the first support portion 281. For example, the light emitting portion 22 may be disposed on the first support portion 281 to face the first surface 10A. The first light receiving portion 24 may be disposed on the second support portion 282. For example, the first light receiving portion 24 may be disposed on the second support portion 282 to face the first surface 10A. The second light receiving portion 26 may be disposed on the third support portion 283. For example, the second light receiving portion 26 may be disposed on the third support portion 283 to face the first surface 10A.

In an embodiment, the first support portion 281 may be positioned between the second support portion 282 and the third support portion 283. The first connection portion 284 may extend from the first support portion 281 to the second support portion 282. The first connection portion 284 may connect the first support portion 281 and the second support portion 282. The second connection portion 285 may extend from the first support portion 281 to the third support portion 283. The second connection portion 285 may connect the first support portion 281 and the third support portion 283. The first connection portion 284 may extend from one side of the first support portion 281, and the second connection portion 285 may extend from the other side of the first support portion 281. For example, the first support portion 281 may be positioned between the first connection portion 284 and the second connection portion 285.

In an embodiment, the substrate 28 may include a flexible printed circuit board or a rigid-flexible printed circuit board. In a case that the substrate 28 includes the rigid-flexible printed circuit board, for example, the first connection portion 284 and the second connection portion 285 may be formed as a flexible portion, and the first support portion 281, the second support portion 282, and the third support portion 283 may be formed as a rigid portion. However, it is not limited by the above-described example. In an embodiment, the substrate 28 may be at least partially curved. For example, the substrate 28 may include a bent portion to correspond to a curvature of the first surface 10A having the ring shape.

In an embodiment, the second support portion 282 may be inclined with respect to the first support portion 281. For example, the second support portion 282 may be inclined with respect to the first support portion 281 such that the first light receiving portion 24 faces a second direction D2 different from a first direction D1 of the light emitting portion 22. In an embodiment, the third support portion 283 may be inclined with respect to the first support portion 281. For example, the third support portion 283 may be inclined with respect to the first support portion 281 such that the second light receiving portion 26 faces a third direction D3 different from the first direction D1 of the light emitting portion 22.

In an embodiment, the light emitting portion 22 may be positioned between the first light receiving portion 24 and the second light receiving portion 26. The light emitting portion 22, the first light receiving portion 24, and the second light receiving portion 26 may be spaced apart. The light emitting portion 22 may face the first area 31 of the insulating member 12. The light emitting portion 22 may be aligned to the first area 31 of the insulating member 12. The light emitting portion 22 may overlap the first area 31. The first light receiving portion 24 may face the second area 32 of the insulating member 12. The first light receiving portion 24 may be aligned to the second area 32 of the insulating member 12. The first light receiving portion 24 may overlap the second area 32. The second light receiving portion 26 may face the third area 33 of the insulating member 12. The second light receiving portion 26 may be aligned to the third area 33 of the insulating member 12. The second light receiving portion 26 may overlap the third area 33.

The light emitting portion 22, the first light receiving portion 24, and the second light receiving portion 26 may be arranged in different directions according to the curvature of the first surface 10A. For example, the light emitting portion 22 may face the first direction D1 toward a center C1 of the hole 15. The first light receiving portion 24 may be inclined with respect to the light emitting portion 22 to face the second direction D2 different from the first direction D1. The second light receiving portion 26 may be inclined with respect to the light emitting portion 22 and the first light receiving portion 24 to face the third direction D3 different from the first direction D1 and the second direction D2. An angle a1 between the first direction D1 and the second direction D2 and an angle a2 between the first direction D1 and the third direction D3 may be, for example, an acute angle, but is not limited thereto. In order for the light from the light emitting portion 22 to be reflected from the body 2 and uniformly received by the first light receiving portion 24 and the second light receiving portion 26, the angle a1 and the angle a2 may be substantially the same, but are not limited thereto. An intersection point between the first direction D1 and the second direction D2 and/or an intersection point between the first direction D1 and the third direction D3 may be the center C1 of the hole 15, but is not limited thereto.

In an embodiment, the light emitting portion 22 may be configured to emit light toward the body 2 of the user. The light emitted from the light emitting portion 22 may pass through the insulating member 12 and be transmitted to the body 2 of the user. The light from the light emitting portion 22 may be reflected from the body 2 of the user. The light reflected from the body 2 of the user may reach the first light receiving portion 24 and the second light receiving portion 26 by passing through the insulating member 12. The first light receiving portion 24 and the second light receiving portion 26 may receive light incident from an outside through the insulating member 12.

In an embodiment, the wearable device 1 may obtain biometric information of the user using the sensor module 20. The sensor module 20 may detect the biometric information of the user based on the light emitted from the light emitting portion 22 and the light received from the light receiving portions 24 and 26. For example, the biometric information may include heart rate and/or saturation of percutaneous oxygen (SpO2), but is not limited thereto. In an embodiment, the sensor module 20 may include at least one of a heartrate measurement (HRM) sensor using an optical sensor or a photoplethysmography (PPG), and an oxygen saturation sensor, but is not limited thereto. The light emitting portion 22 may include a light emitting diode (LED), and the first light receiving portion 24 and the second light receiving portion 26 may include a photo diode, but are not limited thereto.

In one embodiment, the wearable device 1 may include at least one processor (e.g., a processor 2220 of FIG. 22) that is operatively or communicatively connected to the sensor module 20. The at least one processor may be disposed on the substrate 28 of the sensor module 20 or may be disposed on a printed circuit board independent of the substrate 28.

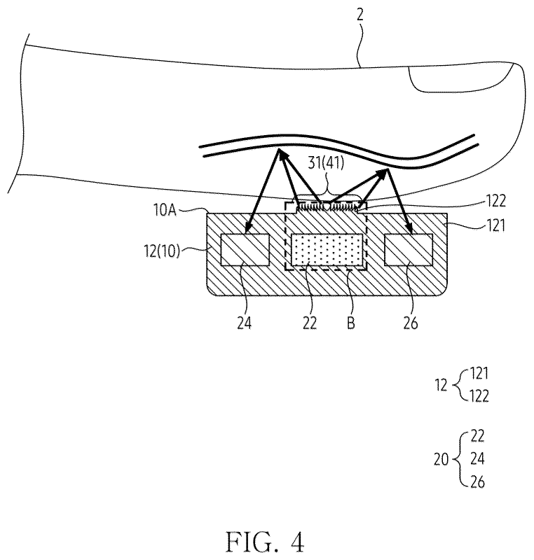

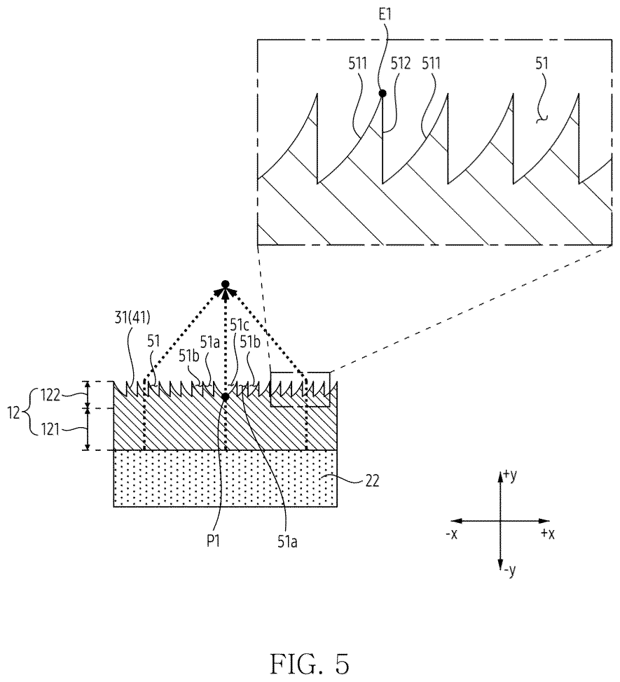

FIG. 4 is a diagram indicating an exemplary wearable device according to an embodiment. FIG. 5 is a diagram for describing an exemplary first pattern according to an embodiment. FIG. 5 is an enlarged view of an area B of FIG. 4. Hereinafter, overlapping descriptions of configurations having the same reference numerals as those described above may be omitted.

Referring to FIGS. 4 and 5, an insulating member 12 according to an embodiment may include a first portion 121 and a second portion 122 on the first portion 121. The first portion 121 may support a sensor module 20 by surrounding the sensor module 20. For example, the first portion 121 may support a light emitting portion 22, a first light receiving portion 24, and a second light receiving portion 26 by surrounding the light emitting portion 22, the first light receiving portion 24, and the second light receiving portion 26. For example, the first portion 121 may fully surround the light emitting portion 22, the first light receiving portion 24, and the second light receiving portion 26. The second portion 122 may extend from the first portion 121. The second portion 122 may be formed on the first portion 121. The second portion 122 may form a first surface 10A together with the first portion 121. The second portion 122 may form a first area 31 in the first surface 10A. The first portion 121 may form a portion of the first surface 10A extending from the first area 31. The first portion 121 and the second portion 122 may be formed of the same material. The first portion 121 and the second portion 122 may have the same chemical component.

The light emitting portion 22 may be aligned with respect to the second portion 122 so that light emitted from the light emitting portion 22 is transmitted to a body 2 of a user through the second portion 122. For example, the light emitting portion 22 may face the second portion 122. The light emitting portion 22 may overlap the second portion 122.

In an embodiment, a pattern may not be formed in an area (e.g., the second area 32 and the third area 33 of FIG. 1) of the first surface 10A overlapping the first light receiving portion 24 and the second light receiving portion 26. In this case, the area of the first surface 10A overlapping the first light receiving portion 24 and the second light receiving portion 26 may be formed as a flat surface or a curved surface having a constant curvature, but is not limited thereto.

In an embodiment, a first pattern 41 may be formed in the second portion 122. For example, the first pattern 41 may be formed in a first area (or the first surface 10A of the second portion 122). The first pattern 41 may be in contact with the body 2 when the user wears a wearable device 1.

In an embodiment, the first pattern 41 may be configured to focus light of the light emitting portion 22 onto the body 2 of the user. For example, the first pattern 41 may include a Fresnel pattern (e.g., a concave Fresnel pattern) that focuses the light emitted from the light emitting portion 22 under the first pattern 41 onto the body of the user. The first pattern 41 may include first grooves 51. Each of or at least one of the first grooves 51 may be formed in an annular shape centered on a first point P1 on the second portion 122 (or the first area 31). The first grooves 51 may be formed such that a radius of each of the first grooves 51 increases as a distance from the first point P1 increases. For example, a groove 51a of the first grooves 51 may have a larger radius than a groove 51b.

In an embodiment, each of the first grooves 51 may include an inclined surface 511 and a surface 512. The inclined surface 511 may extend away from both the first point P1 of the second portion 122 and the light emitting portion 22. For example, the inclined surface 511 positioned in a+x direction based on the first point P1 may extend in a direction between the +x direction and a +y direction. For example, the inclined surface 511 positioned in a −x direction based on the first point P1 may extend in a direction between the −x direction and the +y direction. The surface 512 may extend from a periphery E1 of the inclined surface 511 farthest from the first point P1 toward the light emitting portion 22. For example, the surface 512 may extend in a −y direction from the periphery E1 of the inclined surface 511, but is not limited thereto. The surface 512 may extend between two adjacent inclined surfaces 511. For example, a surface of the groove 51a may extend from an inclined surface of the groove 51a to an inclined surface of the groove 51b. The inclined surface 511 of each of the first grooves 51 may be formed to be concave, but is not limited thereto. For example, the inclined surface 511 of at least some of the first grooves 51 may also be formed to be substantially flat. For example, the inclined surface 511 of at least some of the first grooves 51 may also be formed to be convex. For example, the inclined surface 511 of each of the first grooves 51 may include a concave shape, a convex shape, a substantially flat shape, or a combination shape thereof. The first grooves 51 may be or correspond to a first series of concentric annular grooves. The first point P1 may be a center point of the first area 31, but is not limited thereto. In an embodiment, the first pattern 41 may include a concave portion 51c formed by the inclined surface 511 extending from the first point P1. The concave portion 51c may be positioned at a center of the first pattern 41, but is not limited thereto. Each of the first grooves 51 may surround the concave portion 51c.

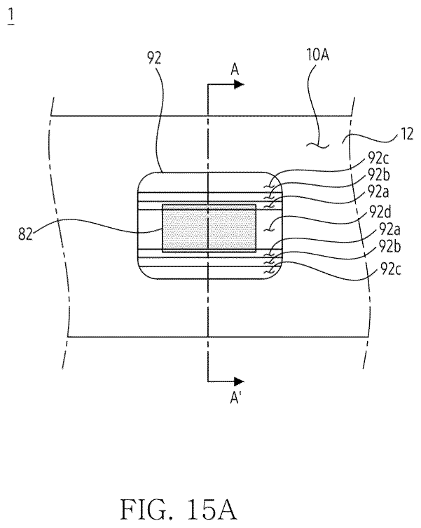

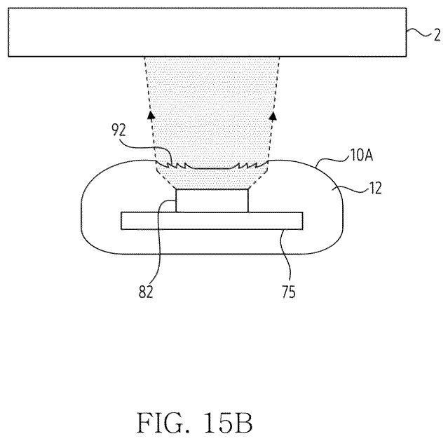

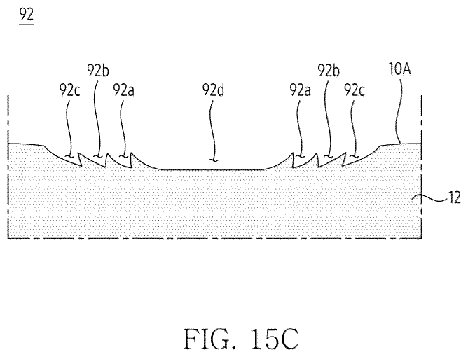

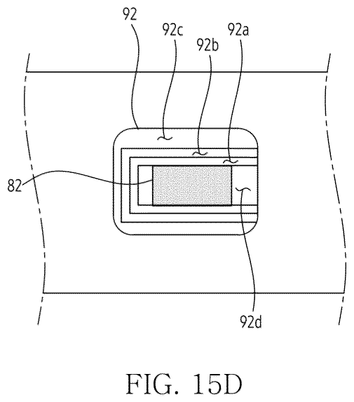

In an embodiment, the first pattern 41 is described as being formed by the annular first grooves 51 surrounding the concave portion 51c, but is not limited thereto. For example, as illustrated in FIG. 15A, the concave portion 51c and each of the first grooves 51 may extend in parallel.

In an embodiment, the first pattern 41 of the first portion 121 may be formed by injecting the insulating member 12 through a mold having a shape corresponding to a shape of the first pattern 41, but is not limited thereto. For example, the first pattern 41 of the first portion 121 may also be formed by processing the insulating member 12 using a machine tool such as a computerized numerical control (CNC).

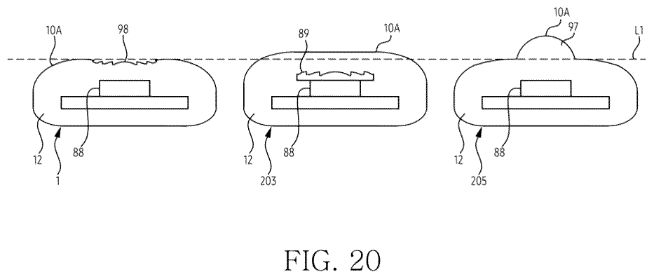

In an embodiment, the light emitted from the light emitting portion 22 may pass through the first portion 121 and the second portion 122 of the insulating member 12. The light emitted from the light emitting portion 22 may be refracted while passing through the first pattern 41 of the second portion 122 and focused onto the body 2 of the user. In a case that the first area 31 is formed flat rather than the first pattern 41, accuracy of a signal obtained may be degraded. In a case of increasing output of the light emitting portion, there is a problem that power consumption is increased. In a case that a concave lens shape is formed in the second portion 122 to focus the light of the light emitting portion onto the body of the user, there is a problem that a thickness of the second portion 122 required to implement the same optical characteristics as the first pattern 41 increases and a size of the wearable device 1 increases. In addition, the concave lens shape may cause a sense of heterogeneity or a pain to the user when worn, and is also not desirable in design. An independent lens member (e.g., a lens 89 of FIG. 20) having the same function as the first pattern 41 may be inserted into the insulating member 12, but may not be applied due to characteristics of the wearable device 1 that should have a thin thickness. On the other hand, in an embodiment, by focusing the light of the light emitting portion 22 with the first pattern 41, efficiency of the sensor module 20 may be improved without increasing the size and the power consumption of the wearable device 1. This may be because the light emitting portion 22 may intensively dimming at a position where reception efficiency of the light receiving portions 24 and 26 is increased through the first pattern 41. In addition, since the first grooves 51 of the first pattern 41 have a fine size, influence on wearability of the user and design may be reduced. The first grooves 51 having the fine size may cause the user to wear the wearable device 1 at an appropriate angle for sensing by providing tactile information to the user. In addition, the first pattern 41 may collect the light of the light emitting portion 22 on the body 2 of the user, thereby reducing an amount of light leaked to an outside (not the body 2 of the user) of the wearable device 1.

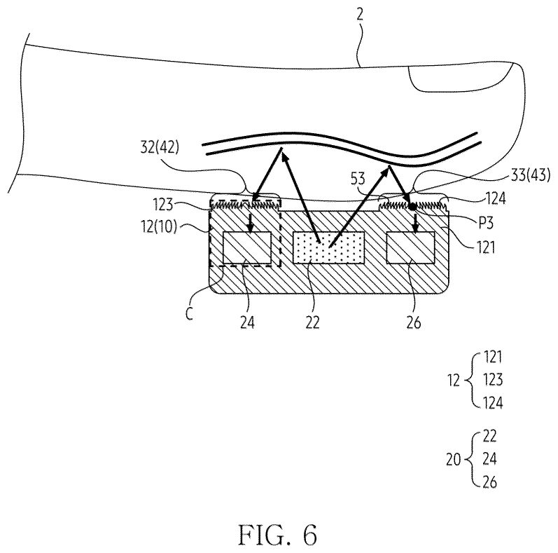

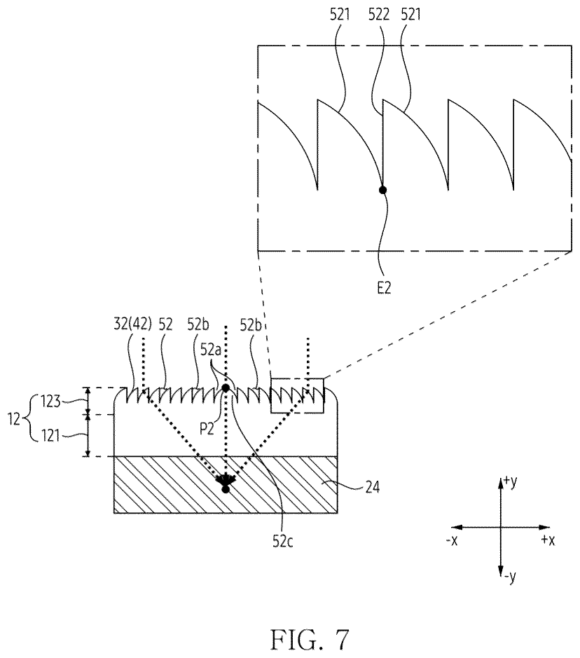

FIG. 6 is a diagram indicating an exemplary wearable device according to an embodiment. FIG. 7 is a diagram for describing an exemplary second pattern according to an embodiment. FIG. 7 is an enlarged view of an area C of FIG. 6.

Referring to FIGS. 6 and 7, an insulating member 12 according to an embodiment may include a third portion 123 and a fourth portion 124 on a first portion 121. The third portion 123 may extend from the first portion 121. The third portion 123 may be formed on the first portion 121. The third portion 123 may form a first surface 10A together with the first portion 121. The third portion 123 may form a second area 32 in the first surface 10A. The fourth portion 124 may be spaced apart from the third portion 123. The fourth portion 124 may extend from the first portion 121. The fourth portion 124 may be formed on the first portion 121. The fourth portion 124 may form the first surface 10A together with the first portion 121 and the third portion 123. The fourth portion 124 may form a third area 33 in the first surface 10A. The first portion 121 may form a portion of the first surface 10A extending from a periphery of the second area 32 and the third area 33. The first portion 121, the third portion 123, and the fourth portion 124 may be formed of the same material. The first portion 121, the third portion 123, and the fourth portion 124 may have the same chemical component.

In an embodiment, a first light receiving portion 24 may be aligned with respect to the third portion 123 so that light reflected from a body 2 of a user is received by the first light receiving portion 24 through the third portion 123. For example, the first light receiving portion 24 may face the third portion 123. The first light receiving portion 24 may overlap the third portion 123.

In an embodiment, a second light receiving portion 26 may be aligned with respect to the fourth portion 124 so that the light reflected from the body 2 of the user is received by the second light receiving portion 26 through the fourth portion 124. For example, the second light receiving portion 26 may face the fourth portion 124. The second light receiving portion 26 may overlap the fourth portion 124.

In an embodiment, a pattern may not be formed in an area (e.g., the first area 31 of FIG. 1) of the first surface 10A overlapping a light emitting portion 22. In this case, the area of the first surface 10A overlapping the light emitting portion 22 may be formed as a flat surface or a curved surface having a constant curvature, but is not limited thereto.

In an embodiment, a second pattern 42 may be formed in the third portion 123. For example, the second pattern 42 may be formed in the second area 32 of the third portion 123 (or the first surface 10A of the third portion 123). The second pattern 42 and a third pattern 43 may be in contact with the body 2 when the user wears a wearable device 1.

In an embodiment, the second pattern 42 may be configured to focus the light reflected from the body of the user and incident on the third portion 123 onto the first light receiving portion 24. For example, the second pattern 42 may include a Fresnel pattern (e.g., a convex Fresnel pattern) that focuses the light incident on the second area 32 onto the first light receiving portion 24. The second pattern 42 may include second grooves 52. Each of the second grooves 52 may be formed in an annular shape centered on a second point P2 on the third portion 123 (or the second area 32). The second grooves 52 may be formed such that a radius of each of the second grooves 52 increases as a distance from the second point P2 increases. For example, a groove 52a of the second grooves 52 may have a larger radius than a groove 52b.

In an embodiment, each of the second grooves 52 may include an inclined surface 521 and a surface 522. The inclined surface 521 may extend away from the second point P2 of the third portion 123 and extend closer to the first light receiving portion 24. For example, the inclined surface 521 positioned in a +x direction based on the second point P2 may extend in a direction between the +x direction and a −y direction. For example, the inclined surface 521 positioned in a −x direction based on the second point P2 may extend in a direction between the −x direction and the −y direction. The surface 522 may extend away from the first light receiving portion 24 from a periphery E2 of the inclined surface 521 farthest from the second point P2. For example, the surface 522 may extend in a +y direction from the periphery E2 of the inclined surface 521, but is not limited thereto. The surface 522 may extend between two adjacent inclined surfaces 521. For example, a surface of the groove 52a may extend from an inclined surface of the groove 52a to an inclined surface of the groove 52b. The inclined surface 521 of each of the second grooves 52 may be formed to be convex, but is not limited thereto. For example, the inclined surface 521 of at least some of the second grooves 52 may be formed to be substantially flat. For example, the inclined surface 521 of the at least some of the second grooves 52 may be formed to be concave. For example, the inclined surface 521 of each of the second grooves 52 may include a concave shape, a convex shape, a substantially flat shape, or a combination shape thereof. The second grooves 52 may be or correspond to a second series of concentric annular grooves. The second point P2 may be a center point of the second area 32, but is not limited thereto. In an embodiment, the second pattern 42 may include a convex portion 52c formed by the inclined surface 521 extending from the second point P2. The convex portion 52c may be positioned at a center of the second pattern 42, but is not limited thereto. Each of the second grooves 52 may surround the convex portion 52c.

In an embodiment, the second pattern 42 is described as being formed by the annular second grooves 52 surrounding the convex portion 52c, but is not limited thereto. For example, as illustrated in FIG. 15A, the convex portions 52c and each of the second grooves 52 may extend in parallel.

In an embodiment, the second pattern 42 of the first portion 121 may be formed by injecting the insulating member 12 through a mold having a shape of the second pattern 42, but is not limited thereto. For example, the second pattern 42 of the first portion 121 may also be formed by processing the insulating member 12 using a machine tool such as a CNC.

In an embodiment, the light reflected from the body 2 of the user may pass through the third portion 123 and the first portion 121 of the insulating member 12. The light reflected from the body 2 of the user may be refracted while passing through the second pattern 42 of the third portion 123 and focused onto the first light receiving portion 24. In a case that the second area 32 is formed flat rather than the second pattern 42, light receiving efficiency of the first light receiving portion 24 may be lowered. In order to improve the light receiving efficiency, when increasing output of the light emitting portion 22, there is a problem that power consumption is increased. In a case that a convex lens shape is formed in the third portion 123 to focus incident light to the light receiving portion, there is a problem in that a thickness of the third portion 123 required to implement the same optical characteristics as the second pattern 42 increases and thereby a size of the wearable device 1 increases. In addition, the convex lens shape may cause a sense of heterogeneity or a pain to the user when worn, and is also not desirable in design. An independent lens member having the same function as the second pattern 42 may be inserted into the insulating member 12, but may not be applied due to characteristics of the wearable device 1 that should have a thin thickness.

On the other hand, in an embodiment, by focusing the light incident on the third portion 123 through the second pattern 42 onto the first light receiving portion 24, efficiency of a sensor module 20 may be improved without increasing a size and power consumption of the wearable device 1, because the first light receiving portion 24 may intensively collect a biometric signal reflected from the body 2 of the user through the second pattern 42. In addition, since the second grooves 52 of the second pattern 42 have a fine size, influence on wearability of the user and design may be reduced. By providing tactile information to the user through the second grooves 52 having the fine size, it may cause the user to wear the wearable device 1 at an appropriate angle for sensing. In addition, the second pattern 42 may collect the light reflected from the body 2 of the user to the first light receiving portion 24, thereby reducing an amount of light leaked to an outside (not the body 2 of the user) of the wearable device 1.

In an embodiment, the third pattern 43 may be formed in the fourth portion 124. For example, the third pattern 43 may be formed in the third area 33 (or the first surface 10A of the fourth portion 124). For the third pattern 43, the description provided with reference to the second pattern 42 may be applied in substantially the same or corresponding manner. For example, the third pattern 43 may be configured to focus the light reflected from the body of the user and incident on the fourth portion 124 onto the second light receiving portion 26. In an embodiment, as the third pattern 43 focuses the light incident on the third portion 123 onto the first light receiving portion 24, the efficiency of the sensor module 20 may be improved without increasing the size and the power consumption of the wearable device 1. The third pattern 43 may include third grooves 53 corresponding to the second grooves 52. For example, each of the third grooves 53 may be formed in an annular shape centered on a third point P3 on the fourth portion 124 (or the third area 33). The third grooves 53 may be formed such that a radius of each of the third grooves 53 increases as a distance from the third point P3 increases. The third grooves 53 of the third pattern 43 may be or correspond to a third series of concentric annular grooves. The third pattern 43 may be substantially the same as the second pattern 42, but is not limited thereto.

The first to third patterns 41, 42, and 43 may be or correspond to first to third optical patterns, first to third condensing patterns, or first to third Fresnel patterns, respectively.

The wearable device 1 according to an embodiment may further include a light receiving portion. In addition, the wearable device 1 may not include either the first light receiving portion 24 or the second light receiving portion 26. For example, the wearable device 1 may not include the second light receiving portion 26, and in this case, the insulating member 12 may not include the fourth portion 124.

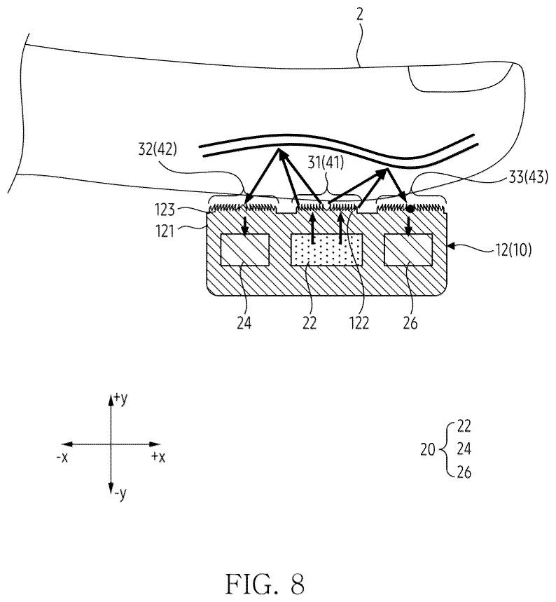

FIG. 8 is a diagram indicating an exemplary wearable device according to an embodiment. Referring to FIG. 8, an insulating member 12 according to an embodiment may include a first portion 121, a second portion 122, a third portion 123, and a fourth portion 124. The second portion 122, the third portion 123, and the fourth portion 124 may be formed on the first portion 121 to be spaced apart from each other. A first pattern 41 of the second portion 122 may have a different pattern from a second pattern 42 of the third portion 123 and a third pattern 43 of the fourth portion 124.

In an embodiment, the first pattern 41 of the second portion 122 may focus light of a light emitting portion 22 onto a body 2 of a user. The second pattern 42 of the third portion 123 may focus the light, which is reflected from the body 2 of the user and incident on, onto a first light receiving portion 24. The third pattern 43 of the fourth portion 124 may focus the light, which is reflected from the body 2 of the user and incident on, onto a second light receiving portion 26. Accordingly, efficiency of a sensor module 20 may be improved.

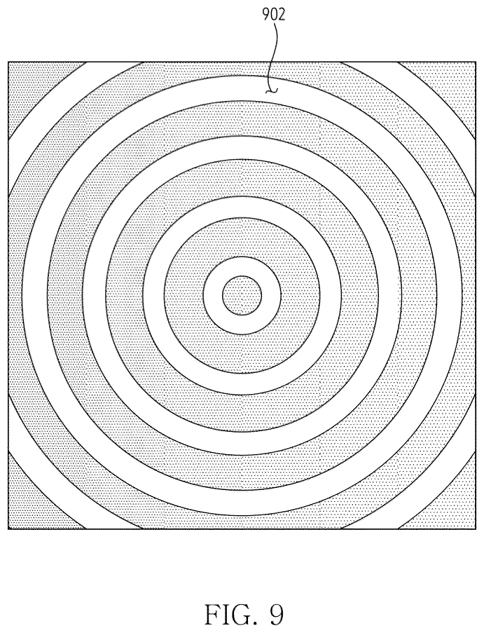

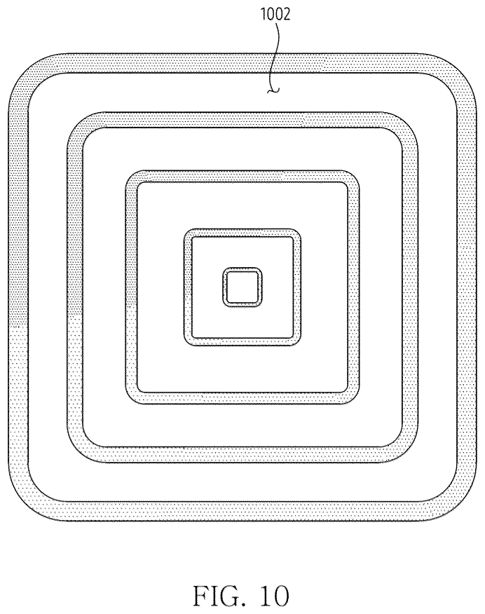

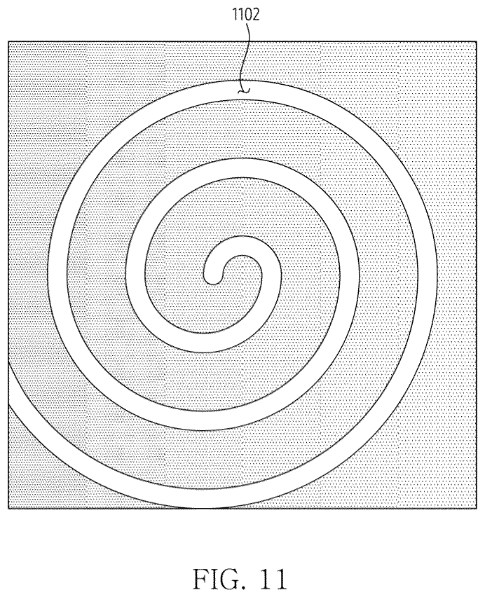

FIGS. 9, 10, and 11 are diagrams indicating patterns of exemplary grooves according to an embodiment. Referring to FIG. 9 together with FIG. 8, grooves 902 may be at least one of first grooves 51 of a first pattern 41, second grooves 52 of a second pattern 42, and third grooves 53 of a third pattern 43. As illustrated in FIG. 9, a shape of each of the grooves 902 may be formed in a circular shape when viewed from above (e.g., when an insulating member 12 is viewed in a −y direction). Referring to FIG. 10 together with FIG. 8, grooves 1002 may be at least one of the first grooves 51 of the first pattern 41, the second grooves 52 of the second pattern 42, and the third grooves 53 of the third pattern 43. As illustrated in FIG. 10, a shape of each of the grooves 1002 may be formed in a square shape (or a square shape with rounded corners) when viewed from above (e.g., when the insulating member 12 is viewed in the −y direction). However, the present disclosure is not limited to the above example embodiment, and various shapes may be probable. For example, referring to FIG. 11 together with FIG. 8, at least one of the first pattern 41, the second pattern 42, and the third pattern 43 may be formed as a spiral groove centered at a point (e.g., the first point P1 of FIG. 5, the second point P2 of FIG. 7, or the third point P3 of FIG. 6) and extending outward therefrom.

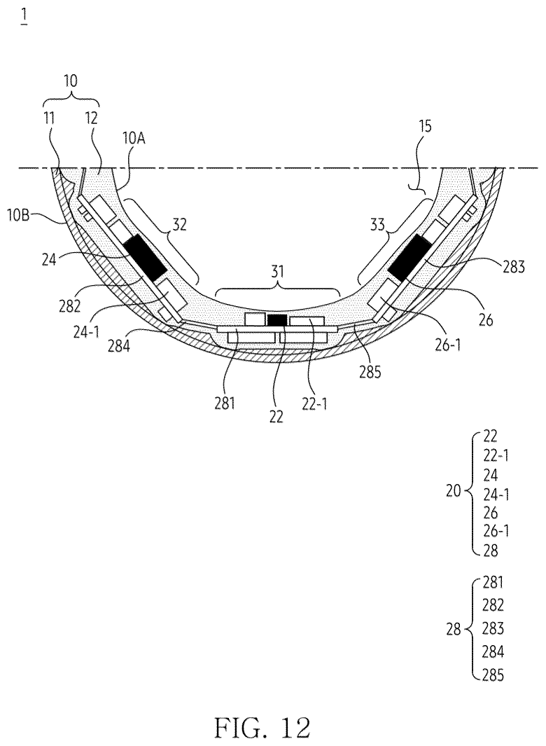

FIG. 12 is a cross-sectional view indicating an exemplary wearable electronic device according to an embodiment. Referring to FIG. 12, the wearable device 1 according to an embodiment may further include a third light receiving portion 22-1, a second light emitting portion 24-1, and a third light emitting portion 26-1. The third light receiving portion 22-1 may perform the same function as the first light receiving portion 24 or the second light receiving portion 26 described above. The second light emitting portion 24-1 and the third light emitting portion 26-1 may perform the same function as the light emitting portion 22 described above.

In an embodiment, the third light receiving portion 22-1 may be disposed on a first support portion 281. The third light receiving portion 22-1 may be positioned adjacent to a light emitting portion 22. The third light receiving portion 22-1 may be a separate unit distinct from the light emitting portion 22, but is not limited thereto. For example, the third light receiving portion 22-1 may be integrated into the light emitting portion 22. For example, the third light receiving portion 22-1 and the light emitting portion 22 may also be implemented on one chip.

In an embodiment, the second light emitting portion 24-1 may be disposed on a second support portion 282. The second light emitting portion 24-1 may be positioned adjacent to a first light receiving portion 24. The second light emitting portion 24-1 may be a separate unit distinct from the first light receiving portion 24, but is not limited thereto. For example, the second light emitting portion 24-1 may be integrated into the first light receiving portion 24. For example, the second light emitting portion 24-1 and the first light receiving portion 24 may also be implemented on one chip.

In an embodiment, the third light emitting portion 26-1 may be disposed on the third support portion 283. The third light emitting portion 26-1 may be positioned adjacent to the second light receiving portion 26. The third light emitting portion 26-1 may be a separate unit distinct from the second light receiving portion 26, but is not limited thereto. For example, the third light emitting portion 26-1 may be integrated into the second light receiving portion 26. For example, the third light emitting portion 26-1 and the second light receiving portion 26 may be implemented on one chip.

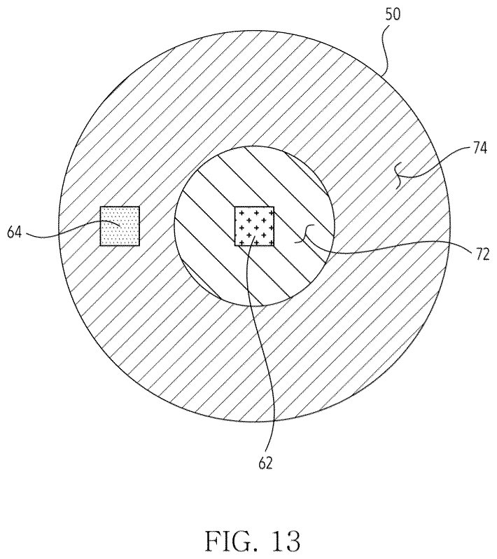

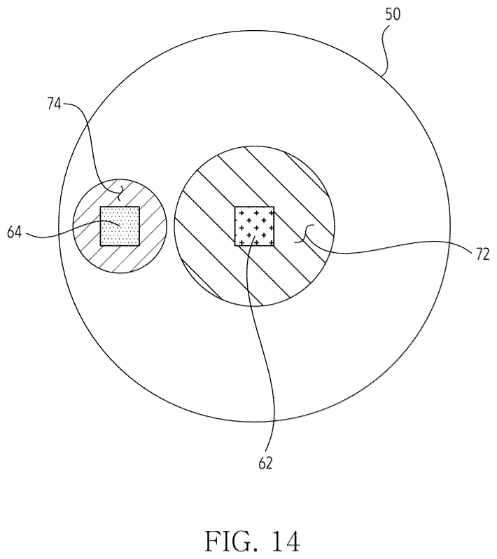

FIGS. 13 and 14 are diagrams for describing a pattern area of a light receiving portion and a light emitting portion according to an embodiment. Referring to FIGS. 13 and 14, a wearable device 1 according to an embodiment may include a first unit 62 and a second unit 64. When the first unit 62 and the second unit 64 are viewed from above, an area 50 may overlap the first unit 62 and the second unit 64. When the first unit 62 and the second unit 64 are viewed from above, the first unit 62 may be closer to a center of the area 50 than the second unit 64. The area 50 may include a first pattern area 72 and a second pattern area 74.

Referring to FIG. 12, the area 50 may include a first area 31, a second area 32, or a third area 33. The first pattern area 72 may include a concave Fresnel pattern (e.g., the first pattern 41 of FIG. 4) and/or a convex Fresnel pattern (e.g., the second pattern 42 of FIG. 6). The second pattern area 74 may include the concave Fresnel pattern and/or the convex Fresnel pattern.

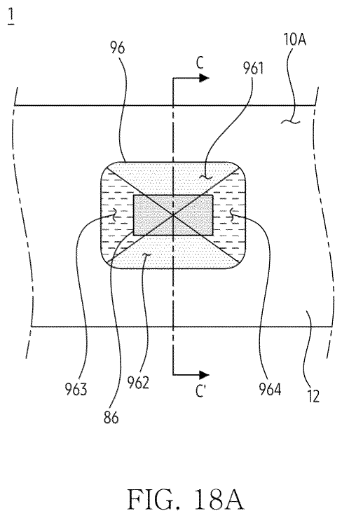

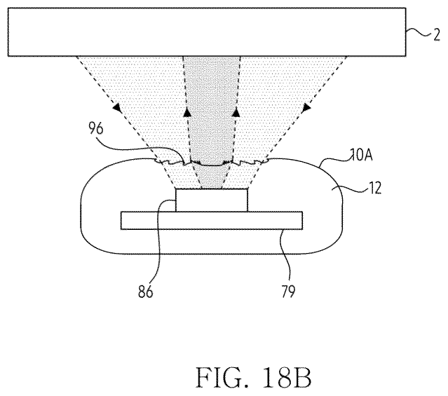

In an embodiment, the first unit 62 may include a light emitting portion 22 and/or a third light receiving portion 22-1, and the second unit 64 may include the third light receiving portion 22-1 and/or the light emitting portion 22. The area 50 may include the first area 31. For example, in a case that the first unit 62 includes the light emitting portion 22, the first pattern area 72 may be formed in the concave Fresnel pattern. In addition, in a case that the second unit 64 includes the third light receiving portion 22-1, the second pattern area 74 may be formed in the convex Fresnel pattern. For another example, in a case that the first unit 62 includes the third light receiving portion 22-1, the first pattern area 72 may be formed in the convex Fresnel pattern. In addition, in a case that the second unit 64 includes the light emitting portion 22, the second pattern area 74 may be formed in the concave Fresnel pattern. For another example, the first unit 62 may include the light emitting portion 22 and the third light receiving portion 22-1. That is, the light emitting portion 22 and the third light receiving portion 22-1 may be formed as one chip integrated into the first unit 62. In this case, the first pattern area 72 may be formed as a composite pattern (e.g., a pattern 96 of FIG. 18A) in which the concave Fresnel pattern and the convex Fresnel pattern are coupled. For example, one area of the first pattern area 72 may be formed as the convex Fresnel pattern, and the remaining area of the first pattern area 72 may be formed as the concave Fresnel pattern. The one area is not limited to one continuous area in the first pattern area 72. In addition, the remaining area is not limited to one continuous area in the first pattern area 72. In a case that the first unit 62 includes the light emitting portion 22 and the third light receiving portion 22-1, the second unit 64 may be omitted, include another light emitting portion, or include another light receiving portion. Corresponding to the other light emitting portion or the other light receiving portion, the second pattern area 74 may also be formed in the concave Fresnel pattern or the convex Fresnel pattern.

In an embodiment, the first unit 62 may include a second light emitting portion 24-1 and/or a first light receiving portion 24, and the second unit 64 may include the first light receiving portion 24 and/or the second light emitting portion 24-1. The area 50 may include the second area 32. For example, in a case that the first unit 62 includes the second light emitting portion 24-1, the first pattern area 72 may be formed in the concave Fresnel pattern. In addition, in a case that the second unit 64 includes the first light receiving portion 24, the second pattern area 74 may be formed in the convex Fresnel pattern. For another example, when the first unit 62 includes the first light receiving portion 24, the first pattern area 72 may be formed in the convex Fresnel pattern. In addition, in a case that the second unit 64 includes a second light emitting portion 24-1, the second pattern area 74 may be formed in the concave Fresnel pattern. For another example, the first unit 62 may include the second light emitting portion 24-1 and the first light receiving portion 24. That is, the second light emitting portion 24-1 and the first light receiving portion 24 may be formed as one chip integrated into the first unit 62. In this case, the first pattern area 72 may be formed in a composite pattern in which the concave Fresnel pattern and the convex Fresnel pattern are coupled. For example, one area of the first pattern area 72 may be formed as the convex Fresnel pattern, and the remaining area of the first pattern area 72 may be formed as the concave Fresnel pattern. The one area is not limited to one continuous area in the first pattern area 72. In addition, the remaining area is not limited to one continuous area in the first pattern area 72. In a case that the first unit 62 includes the second light emitting portion 24-1 and the first light receiving portion 24, the second unit 64 may be omitted, include another light emitting portion, or include another light receiving portion. Corresponding to the other light emitting portion or the other light receiving portion, the second pattern area 74 may also be formed in the concave Fresnel pattern or the convex Fresnel pattern.

In an embodiment, the first unit 62 may include a third light emitting portion 26-1 and/or a second light receiving portion 26, and the second unit 64 may include the second light receiving portion 26 and/or the third light emitting portion 26-1. The area 50 may include the third area 33. For example, in a case that the first unit 62 includes the third light emitting portion 26-1, the first pattern area 72 may be formed in the concave Fresnel pattern. In addition, in a case that the second unit 64 includes the second light receiving portion 26, the second pattern area 74 may be formed in the convex Fresnel pattern. For another example, in a case that the first unit 62 includes the second light receiving portion 26, the first pattern area 72 may be formed in the convex Fresnel pattern. In addition, in a case that the second unit 64 includes the third light emitting portion 26-1, the second pattern area 74 may be formed in the concave Fresnel pattern. For another example, the first unit 62 may include the third light emitting portion 26-1 and the second light receiving portion 26. That is, the third light emitting portion 26-1 and the second light receiving portion 26 may be formed as one chip integrated into the first unit 62. In this case, the first pattern area 72 may be formed in a composite pattern in which the concave Fresnel pattern and the convex Fresnel pattern are coupled. For example, one area of the first pattern area 72 may be formed as the convex Fresnel pattern, and the remaining area of the first pattern area 72 may be formed as the concave Fresnel pattern. The one area is not limited to one continuous area in the first pattern area 72. In addition, the remaining area is not limited to one continuous area in the first pattern area 72. In a case that the first unit 62 includes the third light emitting portion 26-1 and the second light receiving portion 26, the second unit 64 may be omitted, include another light emitting portion, or include another light receiving portion. Corresponding to the other light emitting portion or the other light receiving portion, the second pattern area 74 may also be formed in the concave Fresnel pattern or the convex Fresnel pattern.

As illustrated in FIG. 13, the first pattern area 72 and the second pattern area 74 may form a concentric circle, but the present disclosure is not limited to the above example embodiment. For example, as illustrated in FIG. 14, the first pattern area 72 and the second pattern area 74 may be formed as separate areas in which center points are spaced apart from each other. In a case that the first pattern area 72 and the second pattern area 74 are formed in the concentric circle, a Fresnel pattern of the first pattern area 72 and a Fresnel pattern of the second pattern area 74 may also be formed as patterns that shares the same center point, but are not limited thereto.

The first pattern area 72 and the second pattern area 74 are illustrated to have a circular periphery, but are not limited thereto, and may have a periphery of various shapes (e.g., a polygon such as a triangle and a square).

FIG. 15A is a diagram indicating an exemplary electronic device according to an embodiment. FIG. 15B is a cross-sectional view taken along line A-A′ of FIG. 15A. For convenience of description, a body of a user is also illustrated in FIG. 15B. FIG. 15C is a diagram exemplarily indicating a pattern of FIG. 15A. FIG. 15D is a diagram exemplarily indicating a pattern of FIG. 15A.

Referring to FIG. 15A, a pattern 92 (e.g., the first pattern 41 of FIG. 5, the first pattern area 72 of FIG. 14, or the second pattern area 74 of FIG. 14) may be formed on an insulating member 12 of a wearable device 1 according to an embodiment. For example, the pattern 92 may be formed on a first surface 10A of the wearable device 1 formed by the insulating member 12.

Referring to FIGS. 15A and 15B, the wearable device 1 according to an embodiment may include a substrate 75 (e.g., the substrate 28 of FIG. 12) and a light emitting portion 82. The light emitting portion 82 may be disposed on a support portion (e.g., the first support portion 281, the second support portion 282, or the third support portion 283 of FIG. 12) of the substrate 75. The substrate 75 and the light emitting portion 82 may be positioned in the insulating member 12. The light emitting portion 82 may include, for example, the light emitting portion 22 of FIG. 3, the light emitting portion 22 of FIG. 12, the second light emitting portion 24-1 of FIG. 12, or the third light emitting portion 26-1 of FIG. 12.

In an embodiment, the light emitting portion 82 may be aligned with respect to the pattern 92. For example, the light emitting portion 82 may be positioned under the pattern 92. For example, when viewed from above the first surface 10A, the light emitting portion 82 may be covered by the pattern 92. For example, when viewed from above the first surface 10A, the light emitting portion 82 may overlap the pattern 92. When viewed from above the first surface 10A, an extent of the pattern 92 may be formed larger than an extent of the light emitting portion 82. For example, when viewed from above the first surface 10A, the light emitting portion 82 may be positioned in a periphery of the pattern 92.

In an embodiment, light emitted from the light emitting portion 82 may be focused onto a body 2 (e.g., a blood vessel) of the user by being refracted by the pattern 92.

Referring to FIGS. 15A, 15B, and 15C, the pattern 92 may include a concave portion 92d, first grooves 92a, second grooves 92b, and third grooves 92c. In an embodiment, the concave portion 92d, the first grooves 92a, the second grooves 92b, and the third grooves 92c may extend in parallel with each other. In an embodiment, each of the concave portion 92d, the first grooves 92a, the second grooves 92b, and the third grooves 92c may extend along one direction. For example, the one direction may be a direction extending clockwise or counterclockwise along an inner circumferential surface (e.g., the first surface 10A) of the wearable device 1.

In an embodiment, the concave portion 92d may be positioned between the first grooves 92a. The first grooves 92a may be positioned between the second grooves 92b. The second grooves 92b may be positioned between the third grooves 92c.

In an embodiment, the first grooves 92a may be formed by a first surface (e.g., the surface 412 of FIG. 5) extending from one periphery of the concave portion 92d toward the light emitting portion 82 and/or the substrate 75 and a first inclined surface (e.g., the inclined surface 511 of FIG. 5) extending away from the light emitting portion 82 and/or the substrate 75 from one periphery of the first surface.

In an embodiment, the second grooves 92b may be formed by a second surface (e.g., the surface 412 of FIG. 5) extending from one periphery (e.g., one periphery of the first inclined surface of the first grooves 92a) of the first grooves 92a toward the light emitting portion 82 and/or the substrate 75 and a second inclined surface (e.g., the inclined surface 511 of FIG. 5) extending away from the light emitting portion 82 and/or the substrate 75 from one periphery of the second surface.

In an embodiment, the third grooves 92c may be formed by a third surface (e.g., the surface 412 of FIG. 5) extending from one periphery (e.g., one periphery of the second inclined surface of the second grooves 92b) of the second grooves 92b toward the light emitting portion 82 and/or the substrate 75 and a third inclined surface (e.g., the inclined surface 511 of FIG. 5) extending away from the light emitting portion 82 and/or the substrate 75 from one periphery of the third surface.

In an embodiment, each of the first inclined surface, the second inclined surface, and the inclined surface may include a concave curved surface, a convex curved surface, a flat surface, or a combination shape thereof, similar to the inclined surface 511 of FIG. 5.

In an embodiment, the pattern 92 may include a stripe pattern formed by the concave portion 92d, the first grooves 92a, the second grooves 92b, and the third grooves 92c, but is not limited thereto. For example, referring to FIG. 15D, the first grooves 92a, the second grooves 92b, and the third grooves 92c of the pattern 92 may also be formed in a “U” shape surrounding the concave portion 92d.

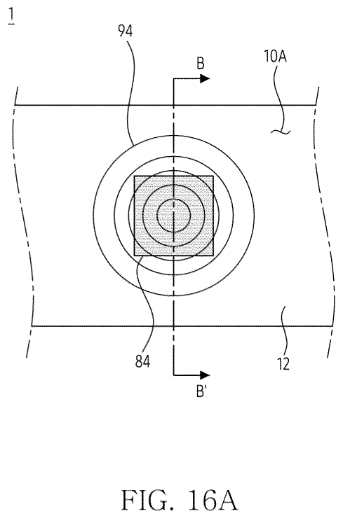

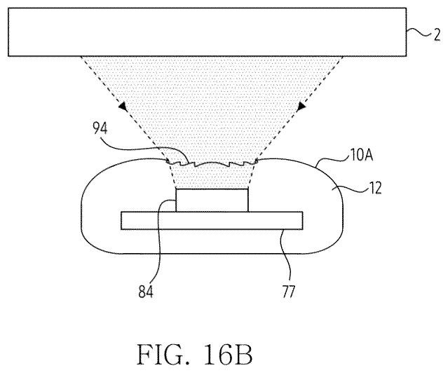

FIG. 16A is a diagram indicating an exemplary electronic device according to an embodiment. FIG. 16B is a cross-sectional view taken along line B-B′ of FIG. 16A. For convenience of description, a body of a user is also illustrated in FIG. 16B.

Referring to FIG. 16A, a pattern 94 (e.g., the second pattern 42 of FIG. 7, the first pattern area 72 of FIG. 14, or the second pattern area 74 of FIG. 14) may be formed on an insulating member 12 of a wearable device 1 according to an embodiment. For example, the pattern 94 may be formed on the first surface 10A of the wearable device 1 formed by the insulating member 12.

Referring to FIGS. 16A and 16B, the wearable device 1 according to an embodiment may include a substrate 77 (e.g., the substrate 28 of FIG. 12) and a light receiving portion 84. The light receiving portion 84 may be disposed on a support portion (e.g., the first support portion 281, the second support portion 282, or the third support portion 283 of FIG. 12) of the substrate 77. The substrate 77 and the light receiving portion 84 may be positioned in the insulating member 12. The light receiving portion 84 may include, for example, the first light receiving portion 24 of FIG. 3, the second light receiving portion 26 of FIG. 3, or the third light receiving portion 22-1 of FIG. 12.

In an embodiment, the light receiving portion 84 may be aligned with respect to the pattern 94. For example, the light receiving portion 84 may be positioned under the pattern 94. For example, when viewed from above the first surface 10A, the light receiving portion 84 may be covered by the pattern 94. For example, when viewed from above the first surface 10A, the light receiving portion 84 may overlap the pattern 94. When viewed from above the first surface 10A, an extent of the pattern 94 may be formed larger than an extent of the light receiving portion 84. For example, when viewed from above the first surface 10A, the light receiving portion 84 may be positioned in a periphery of the pattern 94.

In an embodiment, light reflected from a body 2 (e.g., a blood vessel) of the user may be focused onto the light receiving portion 84 by being refracted by the pattern 94.

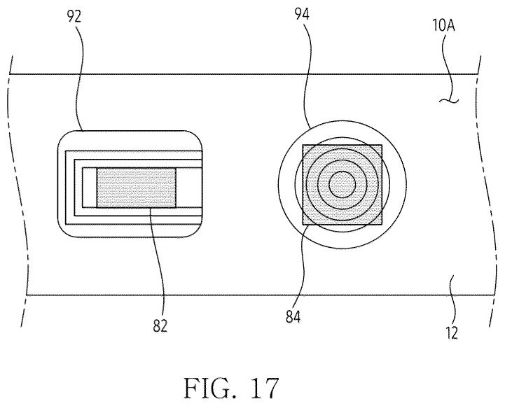

FIG. 17 is a diagram indicating an exemplary electronic device according to an embodiment. Referring to FIG. 17, in an embodiment, patterns 92 and 94 may be spaced apart from each other. Accordingly, a light emitting portion 82 aligned to the pattern 92 and a light receiving portion 84 aligned to the pattern 94 may be spaced apart from each other, but are not limited thereto. For example, the pattern 92 and the pattern 94 are formed in different patterns, but the pattern 92 may also extend continuously from a periphery of the pattern 94, unlike illustration.

FIG. 18A is a diagram indicating an exemplary electronic device according to an embodiment. FIG. 18B is a cross-sectional view taken along line C-C′ of FIG. 18A.

Referring to FIG. 18A, a pattern 96 may be formed on an insulating member 12 of a wearable device 1 according to an embodiment. For example, the pattern 96 may be formed on a first surface 10A of the wearable device 1 formed by the insulating member 12.

In an embodiment, the pattern 96 may include a pattern in which the pattern 92 of FIG. 15A and the pattern 94 of FIG. 16A are combined. For example, a first area 961 and a second area 962 of the pattern 96 may be formed as the pattern 92, and a third area 963 and a fourth area 964 may be formed as the pattern 94. For example, the first area 961 and the second area 962 may be formed in a concave Fresnel pattern, and the third area 963 and the fourth area 964 may be formed in a convex Fresnel pattern.

In an embodiment, the first area 961, the second area 962, the third area 963, and the fourth area 964 may be areas divided into quarters based on two diagonal lines connecting corners of the pattern 96 facing each other, but the division of the area of the pattern 96 is not limited by the illustrated example.

Referring to FIGS. 18A and 18B, the wearable device 1 according to an embodiment may include a substrate 79 (e.g., the substrate 28 of FIG. 12) and a light receiving and emitting portion 86. The light receiving and emitting portion 86 may be disposed on a support portion (e.g., the first support portion 281, the second support portion 282, or the third support portion 283 of FIG. 12) of the substrate 79. The substrate 79 and the light receiving and emitting portion 86 may be positioned in the insulating member 12.

In an embodiment, the light receiving and emitting portion 86 may include a light emitting portion (e.g., the light emitting portion 22 of FIG. 3, the second light emitting portion 24-1 of FIG. 12, or the third light emitting portion 26-1 of FIG. 12) configured to emit light, and a light receiving portion (e.g., the first light receiving portion 24 of FIG. 3, the second light receiving portion 26 of FIG. 3, or the third light receiving portion 22-1 of FIG. 12) configured to receive light incident from an outside. In an embodiment, the light receiving and emitting portion 86 may include one chip including the light emitting portion and the light receiving portion.

In an embodiment, the light receiving and emitting portion 86 may be aligned with respect to the pattern 96. For example, the light receiving and emitting portion 86 may be positioned under the pattern 96. For example, when viewed from above the first surface 10A, the light receiving and emitting portion 86 may be covered by the pattern 96. For example, when viewed from above the first surface 10A, the light receiving and emitting portion 86 may overlap the pattern 96. When viewed from above the first surface 10A, an extent of the pattern 96 may be formed larger than an extent of the light receiving and emitting portion 86. For example, when viewed from above the first surface 10A, the light receiving and emitting portion 86 may be positioned in a periphery of the pattern 96.

In an embodiment, light emitted by the light receiving and emitting portion 86 may be focused onto a body 2 (e.g., a blood vessel) of a user by being refracted by the first area 961 and the second area 962 of the pattern 96. In addition, the light reflected from the body 2 of the user and incident on the insulating member 12 may be focused onto the light receiving and emitting portion 86 by being refracted by the third area 963 and the fourth area 964 of the pattern 96.

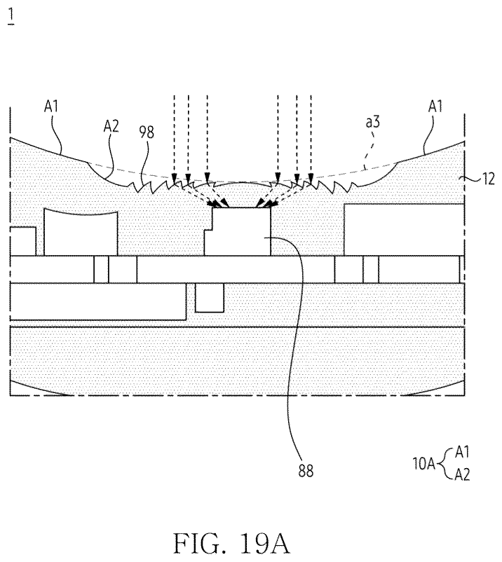

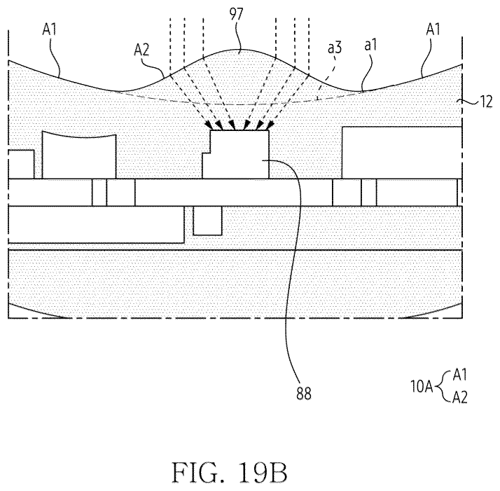

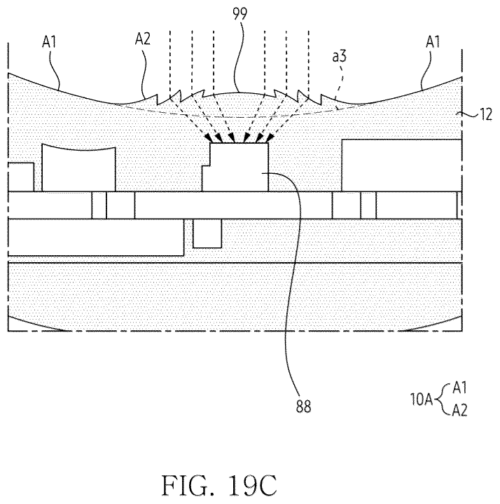

FIG. 19A is a cross-sectional view indicating an exemplary electronic device according to an embodiment. FIG. 19B is a cross-sectional view indicating an electronic device according to a comparative example. FIG. 19C is a cross-sectional view indicating an electronic device according to a comparative example.

Referring to FIG. 19A, a wearable device 1 according to an embodiment may include an optical sensor 88 surrounded by an insulating member 12. The optical sensor 88 may include, for example, a light emitting portion (e.g., the light emitting portion 82 of FIG. 15B), a light receiving portion (e.g., the light receiving portion 84 of FIG. 16B), or a light receiving and emitting portion (e.g., the light receiving and emitting portion 86 of FIG. 18B).

In an embodiment, a pattern 98 covering the optical sensor 88 may be formed on the insulating member 12. For example, the pattern 98 may be formed on a first surface 10A of the insulating member 12. In an embodiment, the pattern 98 may include the pattern 92 of FIG. 15A, the pattern 94 of FIG. 16A, or the pattern 96 of FIG. 18A. In an embodiment, the optical sensor 88 may be aligned with respect to the pattern 98.

In an embodiment, the first surface 10A may include a first region A1 and a second region A2. The first region A1 may surround the second region A2. For example, the first region A1 may define a boundary of the second region A2. The second region A2 may be a surface or an area formed by the pattern 98.

In an embodiment, the first region A1 of the first surface 10A may extend in a circular shape. The second region A2 may be positioned below a virtual region a3 continuous with the first region A1. For example, the second region A2 may be recessed relative to the first region A1. Accordingly, the pattern 98 may be formed without an increase in a thickness of the wearable device 1. That is, without the increase in the thickness of the wearable device 1, efficiency of the optical sensor 88 may be improved by the pattern 98.

In a comparative example of related art, referring to FIG. 19B, a protrusion 97 for improving the efficiency of the optical sensor 88 by refracting light may be formed on the first surface 10A of the insulating member 12. The protrusion 97 of the comparative example may form the second region A2 of the first surface 10A. However, the protrusion 97 may be formed above the virtual region a3 continuous with the first region A1 of the first surface 10A. For example, the protrusion 97 may protrude from the first region A1. That is, in a case that the protrusion 97 is applied to improve the efficiency of the optical sensor 88, the thickness of the wearable device 1 may be increased. In addition, due to the protrusion 97, wearability of the wearable device 1 may be impaired.

In a comparative example, referring to FIG. 19C, a pattern 99 for improving the efficiency of the optical sensor 88 by refracting light may be formed on the first surface 10A of the insulating member 12. The pattern 99 may form the second region A2 of the first surface 10A. The pattern 99 of the comparative example may not be finer than the pattern 98 of FIG. 19A. For example, the pattern 99 of the comparative example may have a smaller number of grooves per unit extent or a smaller width (or height) of each of the grooves than the pattern 98 of FIG. 19A. Therefore, the pattern 99 of the comparative example should be formed thicker than the pattern 98 to satisfy the same optical characteristics. Accordingly, the pattern 99 may be formed above the virtual region a3 continuous with the first region A1 of the first surface 10A. For example, the pattern 99 may protrude from the first region A1. That is, in a case that a non-fine pattern 99 is applied to improve the efficiency of the optical sensor 88, the thickness of the wearable device 1 may be increased. In addition, due to the pattern 99, the wearability of the wearable device 1 may be impaired.

FIG. 20 is a diagram indicating an example of an electronic device according to an embodiment and electronic devices according to a comparative example. In FIG. 12, the wearable device 1 according to an embodiment and the electronic devices 203 and 205 according to a comparative example are both illustrated. In FIG. 12, the optical sensors 88 of the electronic devices 1, 203, and 205 are illustrated in relatively the same position and indicate a virtual line L1 in contact with the first surface 10A of the wearable device 1 to compare heights of the first surfaces 10A of the electronic devices 1, 203, and 205.

The electronic device 203 of the comparative example may include a lens 89 disposed on the optical sensor 88. The wearable device 1 according to an embodiment may not include a lens to which the optical sensor 88 is aligned and distinct from an insulating member 12, such as the lens 89. The lens 89 may be positioned in the insulating member 12. However, since the lens 89 is an independent component of the insulating member 12, a thickness of the insulating member 12 required to stably support the lens 89 by covering the lens 89 may be increased. As a result, a thickness is increased as if the first surface 10A of the electronic device 203 being positioned higher than the virtual line L1.

Like the electronic device 205 of the comparative example, the protrusion 97 for improving the efficiency of the optical sensor 88 necessarily protrudes on the first surface 10A. Accordingly, the thickness of the electronic device 205 is increased as a portion where the protrusion 97 is formed is positioned higher than the virtual line L1.

On the other hand, a pattern 98 of the wearable device 1 according to an embodiment may improve the efficiency of the optical sensor 88 without causing the increase in the thickness of the wearable device 1 or a decrease in wearability. This may be because the pattern 98 of the wearable device 1 according to an embodiment is directly formed on the insulating member 12, unlike the lens 89. In addition, the pattern 98 of wearable device 1 according to an embodiment may be because, unlike the protrusion 97 or the pattern 98 (FIG. 20), it may include a pattern of a fine structure.

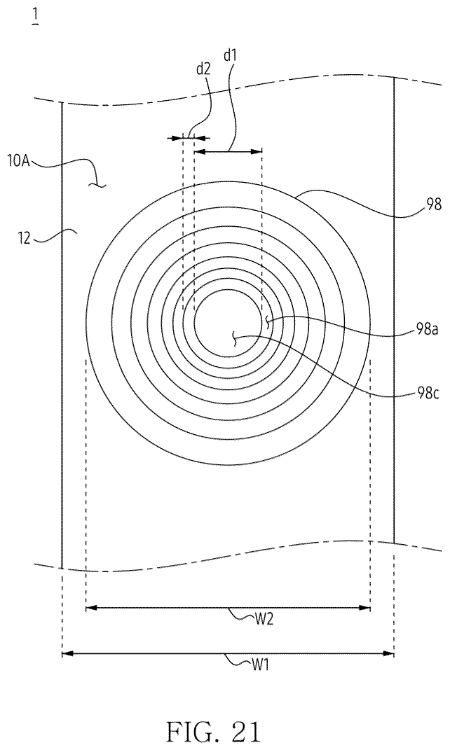

FIG. 21 is a diagram indicating an exemplary electronic device according to an embodiment. Referring to FIG. 21, in an embodiment, a pattern 98 may include a central portion 98c and grooves 98a surrounding the central portion 98c. The central portion 98c may include, for example, the concave portion 51c of FIG. 5, the convex portion 52c of FIG. 7, or the concave portion 92d of FIG. 15C.

For a non-limiting example, the pattern 98 formed on a first surface 10A of an insulating member 12 may be formed by a computerized numerical control (CNC) machining or a molding process using silicon. For a non-limiting example, to remove an air bubble in the insulating member 12 when forming the pattern 98, the molding process may be performed in a pressure chamber.

In an embodiment, a width W2 of the pattern 98 may be smaller than a width W1 of a wearable device 1. The width W1 and the width W2 may be a length based on a direction perpendicular to a direction (e.g., clockwise or counterclockwise) in which the first surface 10A extends in a substantial circular shape. The width W2 of the pattern 98 may be defined by a width d1 of the central portion 98c of the pattern 98 and a width d2 of the grooves 98a.

For example, in a case that the width W1 of the wearable device 1 is approximately 7 mm, the width W2 of the pattern 98 for the required optical characteristics may be approximately 4 mm. In this case, since the CNC machining and the molding process using silicon have a high resolution (e.g., 0.01 mm or less), the central portion 98c having the width d1 of approximately 1 mm and seven grooves 98a having the width d2 of approximately 0.2 mm may be formed in the pattern 98 having the width W2 of approximately 4 mm. However, a specific shape of the pattern 98 is not limited by the above-described numerical example. For example, the width d2 of the grooves 98a may also be formed differently, respectively. In addition, the shape of the pattern 98 may vary according to the width W1 of the wearable device 1, a distance from a sensor, or the required sensor performance.

A wearable device (e.g., the wearable device 1 of FIG. 1) according to an embodiment may include a housing (e.g., the housing 10 of FIG. 4), a sensor module (e.g., the sensor module 20 of FIG. 4) in the housing, and an insulating member (e.g., the insulating member 12 of FIG. 4). The sensor module may include a light emitting portion (e.g., the light emitting portion 22 of FIG. 4) and a light receiving portion (e.g., the first light receiving portion 24 of FIG. 4). The insulating member may include a first portion (e.g., the first portion 121 of FIG. 4) and a second portion (e.g., the second portion 122 of FIG. 4) on the first portion. The first portion may support the light emitting portion and the light receiving portion by surrounding the light emitting portion and the light receiving portion. A Fresnel pattern (e.g., the first pattern 41 of FIG. 4) may be formed in the second portion. The light emitting portion may be aligned to the second portion. The first portion and the second portion in which the Fresnel pattern is formed may be in contact with a portion (e.g., the body 2 of FIG. 4) of a body of a user wearing the wearable device.

In an embodiment, the Fresnel pattern of the second portion may focus light from the light emitting portion onto the portion of the body of the user.

In an embodiment, the light receiving portion may receive light reflected from the portion of the body of the user.

In an embodiment, the sensor module may obtain biometric information of the user based on the light received by the light receiving portion.

In an embodiment, the biometric information may include heart rate information or oxygen saturation information of the user.

In an embodiment, the light receiving portion may be a first light receiving portion (e.g., the first light receiving portion 24 of FIG. 4). The sensor module may include a second light receiving portion (e.g., the second light receiving portion 26 of FIG. 4) in the housing. The first portion may support the second light receiving portion by surrounding the second light receiving portion. The light emitting portion may be spaced apart from the first light receiving portion and the second light receiving portion, and may be positioned between the first light receiving portion and the second light receiving portion.

In an embodiment, the first light receiving portion and the second light receiving portion may receive light reflected from the portion of the body of the user through the first portion.

In an embodiment, the Fresnel pattern of the second portion may be a first Fresnel pattern (e.g., the first pattern 41 of FIG. 8). The insulating member may include a third portion (e.g., the third portion 123 of FIG. 8) on the first portion and a fourth portion (e.g., the fourth portion 124 of FIG. 8) on the first portion. A second Fresnel pattern (e.g., the second pattern 42 of FIG. 8) may be formed on the third portion. A third Fresnel pattern (e.g., the third pattern 43 of FIG. 8) may be formed on the fourth portion. The first light receiving portion may be aligned to the third portion, and the second light receiving portion may be aligned to the fourth portion.

In an embodiment, the first Fresnel pattern may have a different pattern from the second Fresnel pattern and the third Fresnel pattern.

In an embodiment, the second Fresnel pattern may be substantially the same as the third Fresnel pattern.

In an embodiment, the first light receiving portion may receive light reflected from the portion of the body of the user through the first portion and the third portion. The second light receiving portion may receive light reflected from the portion of the body of the user through the first portion and the fourth portion.

In an embodiment, the second Fresnel pattern may be configured to focus light incident on the third portion from an outside onto the first light receiving portion. The third Fresnel pattern may be configured to focus light incident on the fourth portion from an outside onto the second light receiving portion.

In an embodiment, the first Fresnel pattern may include first grooves (e.g., the first grooves 51 of FIG. 5). Each of the first grooves may be formed in an annular shape centered on one point (e.g., the first point P1 of FIG. 5) of the second portion. The first grooves may be formed such that a radius of each of the first grooves gradually increases as a distance from the one point of the second portion increases. Each of the first grooves may include a first inclined surface (e.g., the inclined surface 511 of FIG. 5) and a first surface (e.g., the surface 512 of FIG. 5). The first inclined surface may extend away from both the one point of the second portion and the light emitting portion. The first surface may extend from a periphery (e.g., the periphery E1 of FIG. 5) of the first inclined surface farthest from the one point of the second portion toward the light emitting portion. The second Fresnel pattern may include second grooves (e.g., the second grooves 52 of FIG. 7). Each of the second grooves may be formed in an annular shape centered on one point (e.g., the second point P2 of FIG. 7) of the third portion. The second grooves may be formed such that a radius of each of the second grooves gradually increases as a distance from the one point of the third portion increases. Each of the second grooves may include a second inclined surface (e.g., the inclined surface 521 of FIG. 7) and a second surface (e.g., the surface 522 of FIG. 7). The second inclined surface may extend away from the one point of the third portion and extend closer to the first light receiving portion. The second surface may extend from a periphery (e.g., the periphery E2 of FIG. 7) of the second inclined surface, farthest from the one point of the third portion, in a direction away from the first light receiving portion.

In an embodiment, a shape of each of the first grooves may be formed in a circular shape (e.g., the grooves 902 of FIG. 9) or a rectangular shape (e.g., the grooves 1002 of FIG. 10) when the second portion is viewed from above.

In an embodiment, the Fresnel pattern may include a spiral groove extending away from a first point (e.g., the first point P1 of FIG. 5) of the second portion.

A wearable device (e.g., the wearable device 1 of FIG. 1) according to an embodiment may include a housing (e.g., the housing 10 of FIG. 6), a sensor module (e.g., the sensor module 20 of FIG. 6) in the housing, and an insulating member (e.g., the insulating member 12 of FIG. 6). The sensor module may include a light emitting portion (e.g., the light emitting portion 22 of FIG. 6) and a light receiving portion (e.g., the first light receiving portion 24 of FIG. 6). The insulating member may include a first portion (e.g., the first portion 121 of FIG. 6) and a second portion (e.g., the third portion 123 of FIG. 6) on the first portion. The first portion may support the light emitting portion and the light receiving portion by surrounding the light emitting portion and the light receiving portion. A Fresnel pattern (e.g., the second pattern 42 of FIG. 6) may be formed on the second portion. The light receiving portion may be aligned to the second portion. The first portion and the second portion in which the Fresnel pattern is formed may be in contact with a portion (e.g., the body 2 of FIG. 6) of a body of a user wearing the wearable device.

In an embodiment, the Fresnel pattern of the second portion may be configured to focus light incident on the second portion from an outside onto the light receiving portion.

In an embodiment, the light receiving portion may be a first light receiving portion (e.g., the first light receiving portion 24 of FIG. 6). The sensor module may include a second light receiving portion (e.g., the second light receiving portion 26 of FIG. 6) in the housing. The first portion may support the second light receiving portion by surrounding the second light receiving portion. The light emitting portion may be spaced apart from the first light receiving portion and the second light receiving portion, and may be positioned between the first light receiving portion and the second light receiving portion.

In an embodiment, the Fresnel pattern of the second portion may be a first Fresnel pattern (e.g., the second pattern 42 of FIG. 6). The insulating member may include a third portion (e.g., the fourth portion 124 of FIG. 6) on the first portion. A second Fresnel pattern (e.g., the third pattern 43 of FIG. 6) may be formed on the fourth portion. The second light receiving portion may be aligned to the third portion.

In an embodiment, the first light receiving portion may receive light reflected from the portion of the body of the user through the first portion and the second portion. The second light receiving portion may receive light reflected from the portion of the body of the user through the first portion and the third portion.

A wearable device (e.g., the wearable device 1 of FIG. 1) according to an embodiment may include an insulating member (e.g., the insulating member 12 of FIG. 3) forming a first surface (e.g., the first surface 10A of FIG. 1) that is in contact with a portion (e.g., the body 2 of FIG. 1) of a body of a user wearing the wearable device, and an optical sensor (e.g., the optical sensor 88 of FIG. 19A), supported by being surrounded by the insulating member, the optical sensor configured to detect biometric information of the user. The first surface of the insulating member may include a first region (e.g., the first region A1 of FIG. 19A), and a second region (e.g., the second region A2 of FIG. 19A) surrounded by the first region. The second region may include a Fresnel pattern (e.g., the pattern 98 of FIG. 19A) aligned to the optical sensor and may be recessed relative to the first region. In an embodiment, the optical sensor may include a light emitting portion (e.g., the light emitting portion 22, the second light emitting portion 24-1, and the third light emitting portion 26-1 of FIG. 12, or the light emitting portion 82 of FIG. 15A) configured to emit light toward the first surface. The Fresnel pattern may include a first pattern (e.g., the first pattern 41 of FIG. 4, the pattern 92 of FIG. 15A, the first area 961 of FIG. 18A, or the second area 962 of FIG. 18A) configured to focus the light from the light emitting portion onto a portion of the body of the user. In an embodiment, the optical sensor may include a light receiving portion (e.g., the third light receiving portion 22-1, the first light receiving portion 24, and the second light receiving portion 26 of FIG. 12, or the light receiving portion 84 of FIG. 16A) configured to receive light reflected from a portion of the body of the user. The Fresnel pattern may include a second pattern (e.g., the second pattern 42 and the third pattern 43 of FIG. 6, or the pattern 94 of FIG. 16A) configured to focus light incident on the first surface onto the light receiving portion. In an embodiment, the first region may extend in a substantially circular shape. The second region may be positioned below a virtual region (e.g., the virtual region a3 of FIG. 19A) continuous with the first region.

In an embodiment, the biometric information may include heart rate information or oxygen saturation information.

The wearable device according to an embodiment may include a first light receiving portion (e.g., the first light receiving portion 24 of FIG. 12) supported by being surrounded by the insulating member, and a second light receiving portion (e.g., the second light receiving portion 26 of FIG. 12) supported by being surrounded by the insulating member. The second region may include a second pattern (e.g., the second pattern 42 of FIG. 6 or the pattern 94 of FIG. 16A), aligned to the first light receiving portion, and configured to focus light incident on the first surface onto the first light receiving portion, and a third pattern (e.g., the third pattern 43 of FIG. 6 or the pattern 94 of FIG. 16A) , aligned to the second light receiving portion, and configured to focus light incident on the first surface onto the second light receiving portion.

In an embodiment, the light emitting portion (e.g., the light emitting portion 22 of FIG. 12) may be spaced apart from the first light receiving portion and the second light receiving portion, and may be positioned between the first light receiving portion and the second light receiving portion.

In an embodiment, the optical sensor may include a light receiving portion (e.g., the third light receiving portion 22-1 of FIG. 12 or the light receiving portion 84 of FIG. 16A) configured to receive light reflected from a portion of the body of the user.

In an embodiment, the second region to which the light receiving portion is aligned may include another pattern (e.g., the pattern 94 of FIG. 16A) configured to focus light reflected from a portion of the body of the user onto the light receiving portion. The other pattern may be spaced apart from the first pattern.

In an embodiment, the light receiving portion and the light emitting portion of the optical sensor may be integrated into one chip. The Fresnel pattern may include a fourth pattern (e.g., the third area 963 of FIG. 18A or the fourth area 964 of FIG. 18A) configured to focus light incident on the first surface onto the light receiving portion. The fourth pattern may extend from a periphery of the first pattern.

In an embodiment, the first pattern may have a different pattern from the second pattern, the third pattern, or the fourth pattern.

In an embodiment, the first pattern may include a concave Fresnel pattern. The second pattern, the third pattern, or the fourth pattern may include a convex Fresnel pattern.

In an embodiment, the first light receiving portion may be configured to receive light reflected from a portion of the body of the user through the insulating member. The second light receiving portion may be configured to receive light reflected from a portion of the body of the user through the insulating member.

The wearable device according to an embodiment may not include a lens (e.g., the lens 89 of FIG. 20), aligned to the optical sensor, and distinct from the insulating member.

In an embodiment, the first pattern may include first grooves (e.g., the first grooves 51 of FIG. 5). Each of the first grooves may be formed in an annular shape centered on one point (e.g., the first point P1 of FIG. 5). The first grooves may be formed such that a radius of each of the first grooves gradually increases as a distance from the one point increases. Each of the first grooves may be formed by a first inclined surface (e.g., the inclined surface 511 of FIG. 5) extending away from both the one point and the light emitting portion, and a first surface (e.g., the surface 512 of FIG. 5) extending, from a periphery (e.g., the periphery E1 of FIG. 5) of the first inclined surface farthest from the one point, toward the light emitting portion. The second pattern may include second grooves (e.g., the second grooves 52 of FIG. 7). Each of the second grooves may be formed in an annular shape centered on one point (e.g., the second point P2 of FIG. 7) of the second pattern, the second grooves may be formed such that a radius of each of the second grooves gradually increases as a distance from the one point of the second pattern increases. Each of the second grooves may be formed by a second inclined surface (e.g., the inclined surface 521 of FIG. 7) extending away from the one point of the second pattern and extending closer to the first light receiving portion, and a second surface (e.g., the surface 522 of FIG. 7) extending in a direction away from the first light receiving portion from a periphery (e.g., the periphery E2 of FIG. 7) of the second inclined surface farthest from the one point of the second pattern.

In an embodiment, a shape of each of the first grooves may be formed in a circular shape (e.g., the grooves 902 of FIG. 9) or a rectangular shape (e.g., the grooves 1002 of FIG. 10).

In an embodiment, the first pattern may include a spiral groove (e.g., the grooves 1102 of FIG. 11).

In an embodiment, the first pattern may include a concave portion (e.g., the concave portion 92d of FIG. 15A), first grooves (e.g., the first grooves 92a of FIG. 15A) surrounding the concave portion, second grooves (e.g., the second grooves 92b of FIG. 15A) extending in parallel with the first grooves and surrounding the first grooves, and third grooves (e.g., the third grooves 92c of FIG. 15A) extending in parallel with the second grooves and surrounding the second grooves.

In an embodiment, the concave portion may extend in parallel to the first grooves. Each of the concave portion, the first grooves, the second grooves, and the third grooves may extend clockwise along a circumference of a first surface of the insulating member.

In an embodiment, when viewed from above the first surface, a shape of each of the first grooves, the second grooves, and the third grooves may include a U shape.

In an embodiment, the Fresnel pattern of the insulating member may be formed by a molding process using silicon or a CNC machining.

The wearable device according to an embodiment may include a substrate (e.g., the substrate 28 of FIG. 12) supported by being surrounded by the insulating member. The substrate may include a first support portion (e.g., the first support portion 281 of FIG. 12) on which the optical sensor is disposed, a second support portion (e.g., the second support portion 282 of FIG. 12) on which the second light receiving portion is disposed, a third support portion (e.g., the third support portion 283 of FIG. 12) on which the third light receiving portion is disposed, a first connection portion (e.g., the first connection portion 284 of FIG. 12) connecting the first support portion and the second support portion, and a second connection portion (e.g., the second connection portion 285 of FIG. 12) connecting the first support portion and the third support portion.

FIG. 22 is a block diagram illustrating an electronic device 2201 in a network environment 2200 according to various embodiments. Referring to FIG. 22, the electronic device 2201 in the network environment 2200 may communicate with an electronic device 2202 via a first network 2298 (e.g., a short-range wireless communication network), or at least one of an electronic device 2204 or a server 2208 via a second network 2299 (e.g., a long-range wireless communication network). According to an embodiment, the electronic device 2201 may communicate with the electronic device 2204 via the server 2208. According to an embodiment, the electronic device 2201 may include a processor 2220, memory 2230, an input module 2250, a sound output module 2255, a display module 2260, an audio module 2270, a sensor module 2276, an interface 2277, a connecting terminal 2278, a haptic module 2279, a camera module 2280, a power management module 2288, a battery 2289, a communication module 2290, a subscriber identification module(SIM) 2296, or an antenna module 2297. In some embodiments, at least one of the components (e.g., the connecting terminal 2278) may be omitted from the electronic device 2201, or one or more other components may be added in the electronic device 2201. In some embodiments, some of the components (e.g., the sensor module 2276, the camera module 2280, or the antenna module 2297) may be implemented as a single component (e.g., the display module 2260).

The processor 2220 may execute, for example, software (e.g., a program 2240) to control at least one other component (e.g., a hardware or software component) of the electronic device 2201 coupled with the processor 2220, and may perform various data processing or computation. According to an embodiment, as at least part of the data processing or computation, the processor 2220 may store a command or data received from another component (e.g., the sensor module 2276 or the communication module 2290) in volatile memory 2232, process the command or the data stored in the volatile memory 2232, and store resulting data in non-volatile memory 2234. According to an embodiment, the processor 2220 may include a main processor 2221 (e.g., a central processing unit (CPU) or an application processor (AP)), or an auxiliary processor 2223 (e.g., a graphics processing unit (GPU), a neural processing unit (NPU), an image signal processor (ISP), a sensor hub processor, or a communication processor (CP)) that is operable independently from, or in conjunction with, the main processor 2221. For example, when the electronic device 2201 includes the main processor 2221 and the auxiliary processor 2223, the auxiliary processor 2223 may be adapted to consume less power than the main processor 2221, or to be specific to a specified function. The auxiliary processor 2223 may be implemented as separate from, or as part of the main processor 2221.

The auxiliary processor 2223 may control at least some of functions or states related to at least one component (e.g., the display module 2260, the sensor module 2276, or the communication module 2290) among the components of the electronic device 2201, instead of the main processor 2221 while the main processor 2221 is in an inactive (e.g., sleep) state, or together with the main processor 2221 while the main processor 2221 is in an active state (e.g., executing an application). According to an embodiment, the auxiliary processor 2223 (e.g., an image signal processor or a communication processor) may be implemented as part of another component (e.g., the camera module 2280 or the communication module 2290) functionally related to the auxiliary processor 2223. According to an embodiment, the auxiliary processor 2223 (e.g., the neural processing unit) may include a hardware structure specified for artificial intelligence model processing. An artificial intelligence model may be generated by machine learning. Such learning may be performed, e.g., by the electronic device 2201 where the artificial intelligence is performed or via a separate server (e.g., the server 2208). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 2230 may store various data used by at least one component (e.g., the processor 2220 or the sensor module 2276) of the electronic device 2201. The various data may include, for example, software (e.g., the program 2240) and input data or output data for a command related thereto. The memory 2230 may include the volatile memory 2232 or the non-volatile memory 2234.

The program 2240 may be stored in the memory 2230 as software, and may include, for example, an operating system (OS) 2242, middleware 2244, or an application 2246.

The input module 2250 may receive a command or data to be used by another component (e.g., the processor 2220) of the electronic device 2201, from the outside (e.g., a user) of the electronic device 2201. The input module 2250 may include, for example, a microphone, a mouse, a keyboard, a key (e.g., a button), or a digital pen (e.g., a stylus pen).

The sound output module 2255 may output sound signals to the outside of the electronic device 2201. The sound output module 2255 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 2260 may visually provide information to the outside (e.g., a user) of the electronic device 2201. The display module 2260 may include, for example, a display, a hologram device, or a projector and control circuitry to control a corresponding one of the display, hologram device, and projector. According to an embodiment, the display module 2260 may include a touch sensor adapted to detect a touch, or a pressure sensor adapted to measure the intensity of force incurred by the touch.

The audio module 2270 may convert a sound into an electrical signal and vice versa. According to an embodiment, the audio module 2270 may obtain the sound via the input module 2250, or output the sound via the sound output module 2255 or a headphone of an external electronic device (e.g., an electronic device 2202) directly (e.g., through a wire or wires) or wirelessly coupled with the electronic device 2201.

The sensor module 2276 may detect an operational state (e.g., power or temperature) of the electronic device 2201 or an environmental state (e.g., a state of a user) external to the electronic device 2201, and then generate an electrical signal or data value corresponding to the detected state. According to an embodiment, the sensor module 2276 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 2277 may support one or more specified protocols to be used for the electronic device 2201 to be coupled with the external electronic device (e.g., the electronic device 2202) directly (e.g., through a wire or wires) or wirelessly. According to an embodiment, the interface 2277 may include, for example, a high definition multimedia interface (HDMI), a universal serial bus (USB) interface, a secure digital (SD) card interface, or an audio interface.

A connecting terminal 2278 may include a connector via which the electronic device 2201 may be physically connected with the external electronic device (e.g., the electronic device 2202). According to an embodiment, the connecting terminal 2278 may include, for example, an HDMI connector, a USB connector, an SD card connector, or an audio connector (e.g., a headphone connector).

The haptic module 2279 may convert an electrical signal into a mechanical stimulus (e.g., a vibration or a movement) or electrical stimulus which may be recognized by a user via his tactile sensation or kinesthetic sensation. According to an embodiment, the haptic module 2279 may include, for example, a motor, a piezoelectric element, or an electric stimulator.

The camera module 2280 may capture a still image or moving images. According to an embodiment, the camera module 2280 may include one or more lenses, image sensors, image signal processors, or flashes.

The power management module 2288 may manage power supplied to the electronic device 2201. According to an embodiment, the power management module 2288 may be implemented as at least part of, for example, a power management integrated circuit (PMIC).

The battery 2289 may supply power to at least one component of the electronic device 2201. According to an embodiment, the battery 2289 may include, for example, a primary cell which is not rechargeable, a secondary cell which is rechargeable, or a fuel cell.

The communication module 2290 may support establishing a direct (e.g., wired) communication channel or a wireless communication channel between the electronic device 2201 and the external electronic device (e.g., the electronic device 2202, the electronic device 2204, or the server 2208) and performing communication via the established communication channel. The communication module 2290 may include one or more communication processors that are operable independently from the processor 2220 (e.g., the application processor (AP)) and supports a direct (e.g., wired) communication or a wireless communication. According to an embodiment, the communication module 2290 may include a wireless communication module 2292 (e.g., a cellular communication module, a short-range wireless communication module, or a global navigation satellite system (GNSS) communication module) or a wired communication module 2294 (e.g., a local area network (LAN) communication module or a power line communication (PLC) module). A corresponding one of these communication modules may communicate with the external electronic device via the first network 2298 (e.g., a short-range communication network, such as Bluetooth™, wireless-fidelity (Wi-Fi) direct, or infrared data association (IrDA)) or the second network 2299 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 2292 may identify and authenticate the electronic device 2201 in a communication network, such as the first network 2298 or the second network 2299, using subscriber information (e.g., international mobile subscriber identity (IMSI)) stored in the subscriber identification module 2296.

The wireless communication module 2292 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 2292 may support a high-frequency band (e.g., the mmWave band) to achieve, e.g., a high data transmission rate. The wireless communication module 2292 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 2292 may support various requirements specified in the electronic device 2201, an external electronic device (e.g., the electronic device 2204), or a network system (e.g., the second network 2299). According to an embodiment, the wireless communication module 2292 may support a peak data rate (e.g., 20 Gbps or more) for implementing eMBB, loss coverage (e.g., 2264 dB or less) for implementing mMTC, or U-plane latency (e.g., 0.5 ms or less for each of downlink (DL) and uplink (UL), or a round trip of 22 ms or less) for implementing URLLC.

The antenna module 2297 may transmit or receive a signal or power to or from the outside (e.g., the external electronic device) of the electronic device 2201. According to an embodiment, the antenna module 2297 may include an antenna including a radiating element composed of a conductive material or a conductive pattern formed in or on a substrate (e.g., a printed circuit board (PCB)). According to an embodiment, the antenna module 2297 may include a plurality of antennas (e.g., array antennas). In such a case, at least one antenna appropriate for a communication scheme used in the communication network, such as the first network 2298 or the second network 2299, may be selected, for example, by the communication module 2290 (e.g., the wireless communication module 2292) from the plurality of antennas. The signal or the power may then be transmitted or received between the communication module 2290 and the external electronic device via the selected at least one antenna. According to an embodiment, another component (e.g., a radio frequency integrated circuit (RFIC)) other than the radiating element may be additionally formed as part of the antenna module 2297.

According to various embodiments, the antenna module 2297 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) between the components via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 2201 and the external electronic device 2204 via the server 2208 coupled with the second network 2299. Each of the electronic devices 2202 or 2204 may be a device of a same type as, or a different type, from the electronic device 2201. According to an embodiment, all or some of operations to be executed at the electronic device 2201 may be executed at one or more of the external electronic devices 2202, 2204, or 2208. For example, if the electronic device 2201 should perform a function or a service automatically, or in response to a request from a user or another device, the electronic device 2201, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 2201. The electronic device 2201 may provide the outcome, with or without further processing of the outcome, as at least part of a reply to the request. To that end, a cloud computing, distributed computing, mobile edge computing (MEC), or client-server computing technology may be used, for example. The electronic device 2201 may provide ultra-low-latency services using, e.g., distributed computing or mobile edge computing. In another embodiment, the external electronic device 2204 may include an internet-of-things (IoT) device. The server 2208 may be an intelligent server using machine learning and/or a neural network. According to an embodiment, the external electronic device 2204 or the server 2208 may be included in the second network 2299. The electronic device 2201 may be applied to intelligent services (e.g., smart home, smart city, smart car, or healthcare) based on 5G communication technology or IoT-related technology.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

One or more embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. A singular form of a noun corresponding to an item may include one or more of the things unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” or “connected with” another element (e.g., a second element), it means that the element may be coupled with the other element directly (e.g., through a wire or wires), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 2240) including one or more instructions that are stored in a storage medium (e.g., internal memory 2236 or external memory 2238) that is readable by a machine (e.g., the electronic device 2201). For example, a processor (e.g., the processor 2220) of the machine (e.g., the electronic device 2201) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between a case in which data is semi-permanently stored in the storage medium and a case in which the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

What is claimed is:

1. A wearable device comprising:
an insulating member having a first surface contacting with a portion of a body of a user wearing the wearable device; and
an optical sensor surrounded by the insulating member and configured to detect biometric information of the user,
wherein the first surface includes:
a first region; and
a second region surrounded by the first region, and
wherein the second region includes a Fresnel pattern aligned to the optical sensor and the second region is recessed relative to the first region.

2. The wearable device of claim 1, wherein the optical sensor includes a light emitting portion configured to emit light toward the first surface, and
wherein the Fresnel pattern includes a first pattern focusing the light from the light emitting portion onto a portion of the body of the user.

3. The wearable device of claim 1, wherein the optical sensor includes a light receiving portion configured to receive light reflected from a portion of the body of the user, and
wherein the Fresnel pattern includes a second pattern focusing light incident on the first surface onto the light receiving portion.

4. The wearable device of claim 1, wherein the first region extends in a substantially circular shape, and
wherein the second region is positioned below a plane extending from the first region.

5. The wearable device of claim 1, wherein the biometric information includes heart rate information or oxygen saturation information.

6. The wearable device of claim 2, further comprising:
a first light receiving portion surrounded by the insulating member; and
a second light receiving portion surrounded by the insulating member,
wherein the second region includes:
a second pattern aligned to the first light receiving portion and focusing light incident on the first surface onto the first light receiving portion; and
a third pattern aligned to the second light receiving portion and focusing light incident on the first surface onto the second light receiving portion.

7. The wearable device of claim 6, wherein the light emitting portion is spaced apart from the first light receiving portion and the second light receiving portion, and the light emitting portion is positioned between the first light receiving portion and the second light receiving portion.

8. The wearable device of claim 6, wherein the optical sensor includes a light receiving portion configured to receive light reflected from a portion of the body of the user.

9. The wearable device of claim 8, wherein the second region to which the light receiving portion is aligned includes another pattern focusing light reflected from a portion of the body of the user onto the light receiving portion, and
wherein the another pattern is spaced apart from the first pattern.

10. The wearable device of claim 8, wherein the light receiving portion and the light emitting portion of the optical sensor are integrated into one chip,
wherein the Fresnel pattern includes a fourth pattern focusing light incident on the first surface onto the light receiving portion, and
wherein the fourth pattern extends from a periphery of the first pattern.

11. The wearable device of claim 10, wherein the first pattern has a different pattern from the second pattern, the third pattern, or the fourth pattern.

12. The wearable device of claim 10, wherein the first pattern includes a concave Fresnel pattern, and
wherein the second pattern, the third pattern, or the fourth pattern includes a convex Fresnel pattern.

13. The wearable device of claim 6, wherein the first light receiving portion is configured to receive light reflected from a portion of the body of the user through the insulating member, and
wherein the second light receiving portion is configured to receive light reflected from a portion of the body of the user through the insulating member.

14. The wearable device of claim 1, wherein the wearable device does not include a lens,
wherein the wearable device is aligned to the optical sensor, and
wherein the wearable device is distinct from the insulating member.

15. The wearable device of claim 6, wherein the first pattern includes first grooves,
wherein each of the first grooves is formed in an annular shape centered on one point,
wherein the first grooves are formed such that a radius of each of the first grooves gradually increases as a distance from the one point increases,
wherein each of the first grooves is formed by:
a first inclined surface extending away from both the one point and the light emitting portion; and
a first surface extending, from a periphery of the first inclined surface farthest from the one point, toward the light emitting portion;
wherein the second pattern includes second grooves,
wherein each of the second grooves is formed in an annular shape centered on one point of the second pattern,
wherein the second grooves are formed such that a radius of each of the second grooves gradually increases as a distance from the one point of the second pattern increases,
wherein each of the second grooves is formed by:
a second inclined surface extending away from the one point of the second pattern and extending closer to the first light receiving portion; and
a second surface extending in a direction away from the first light receiving portion from a periphery of the second inclined surface farthest from the one point of the second pattern.

16. The wearable device of claim 15, wherein a shape of each of the first grooves is formed in a circular shape or a rectangular shape.

17. The wearable device of claim 2, wherein the first pattern includes a spiral groove.

18. The wearable device of claim 2, wherein the first pattern includes:
a concave portion;
first grooves surrounding the concave portion;
second grooves extending in parallel with the first grooves and surrounding the first grooves; and
third grooves extending in parallel with the second grooves and surrounding the second grooves.

19. The wearable device of claim 18, wherein the concave portion extends in parallel to the first grooves, and
wherein each of the concave portion, the first grooves, the second grooves, and the third grooves extends in a clockwise direction along a circumference of the first surface of the insulating member.

20. The wearable device of claim 18, wherein, when viewed from above the first surface, a shape of each of the first grooves, the second grooves, and the third grooves includes a U shape.
